(19) United States
(12) Patent Application Publication
(10) Pub. No.: US 2025/0255883 A1
(43) Pub. Date: Aug. 14, 2025

(54) Compositions and Methods for Treating Aging Hair

(71) Applicant: CHL Industries, LLC, Austin, TX (US)

(72) Inventors: John P. McCook, Austin, TX (US); David B. Vasily, Bethlehem, PA (US)

(21) Appl. No.: 19/192,917

(22) Filed: Apr. 29, 2025

Related U.S. Application Data
(63) Continuation-in-part of application No. 17/887,471, filed on Aug. 14, 2022.
(60) Provisional application No. 63/640,607, filed on Apr. 30, 2024.
(60) Provisional application No. 63/233,234, filed on Aug. 14, 2021.

(51) Int. Cl. A61K 31/555; A61K 9/00; A61P 17/14

(52) U.S. Cl. CPC A61K 31/555; A61K 9/0014; A61P 17/14

(57) ABSTRACT

Treatment compositions and methods for treating and/or preventing human hair thinning or hair loss comprising applying a chlorin treatment composition to an area of skin/scalp in need of treatment. A treatment composition preferably comprises a copper chlorin, such as a sodium copper chlorophyllin complex, trisodium copper chlorin p6, disodium copper isochlorin e4, or trisodium copper chlorin e6. A treatment composition comprises around 0.02 to 0.1% by weight of a chlorin, preferably a copper chlorin. A density and/or diameter of hair in an area being treated may be increased by at least 5%, more preferably at least 15%. Immune privilege in an area being treated may also be restored as measured by change in certain immunoglobulin antibodies.

### CROSS-REFERENCE TO RELATED APPLICATIONS

This application claims the benefit of U.S. Provisional Application Ser. No. 63/640,607 filed on Apr. 30, 2024, the disclosure of which is incorporated herein by reference. This application is also a continuation-in-part of U.S. application Ser. No. 17/887,471 filed on Aug. 14, 2022, which claims to the benefit of U.S. Provisional Application Ser. No. 63/233,234 filed on Aug. 14, 2021, the disclosures of which are incorporated herein by reference.

### BACKGROUND OF THE DISCLOSURE

The present disclosure relates, generally, to topically applied compositions and methods for reducing the signs of human hair loss or thinning, particularly age-related loss or thinning of human hair aging, by increasing hair density and/or thickness of individual hairs, particularly terminal hairs on a human scalp.

Hair is a unique characteristic which differentiates mammals from other animals in the animal Kingdom. Interestingly, hair has no vital function in humans, yet its psychological function is extremely important. Loss of hair from the scalp can be extremely distressing, as is the growth of body or facial hair in excess. Human hair serves a wide range of purposes, both practical and aesthetic, and its properties have been the subject of extensive scientific research. Moreover, hair has played a crucial role in cultural and social contexts throughout history, as it often symbolizes various aspects of human identity and social status.

The insulating properties of hair in mammals, particularly for those that inhabit cold environments or reside at high elevations, are well-documented. The ability of hair to trap a layer of air between the skin and the environment is crucial in regulating body temperature, conserving energy, and protecting against environmental stressors.

The human scalp hair serves a critical insulating function, aiding in the regulation of body temperature and preventing heat loss through the head. This feature is particularly crucial for newborns and infants, whose bodies are not yet fully developed to regulate temperature as efficiently as adults. The insulation provided by scalp hair is a critical factor in maintaining body warmth, especially in colder environments or during extreme weather conditions. Indeed, the importance of this insulation function is reflected in the significant cultural and social value that humans attach to their hair, as evidenced by various hair care practices and rituals throughout history.

In addition to its insulation properties, hair in mammals plays a crucial protective role by shielding the skin from environmental factors such as UV radiation, chemicals, and physical damage. For instance, in humans, eyelashes and eyebrows serve as protective barriers, safeguarding the eyes from dust, debris, and sunlight. In some mammals, hair helps deter predators by making them appear more dangerous or larger. Similarly, scalp hair in humans may serve as a protective function by reducing the impact of blows to the head. The protective nature of hair has been the subject of significant scientific research and has led to numerous innovations in materials and technologies that mimic the protective properties of hair. This feature has significant implications for the survival and well-being of mammals in cold climates. As such, hair insulation has garnered significant attention in the scientific community and has been extensively studied for its potential applications in various fields. Indeed, the development of insulation materials that can mimic the properties of hair has been an area of active research and innovation, with several patent applications filed for related technologies.

Hair is a vital aspect of human biology that has played a significant role throughout human history. The importance of hair in humans goes beyond its functional purposes and extends to cultural and aesthetic significance. Hair is often seen as an important aspect of personal identity and expression and is subject to a wide range of cultural practices and beauty standards. For example, hair styles and treatments may vary widely depending on cultural norms and individual preferences. Hair may also be used as a means of self-expression, such as through hair dye or styling.

Hair in humans can be divided into two main types: vellus hair and terminal hair. vellus hair is the fine, soft hair that covers much of the body, while terminal hair is the thicker, darker hair found on the scalp, face, armpits, and pubic area. The thickness and distribution of terminal hair is influenced by hormonal factors and may vary widely depending on sex and age.

Hair growth occurs in cycles, and each human hair follicle (HHF) goes through distinct phases: growth (anagen), apoptosis-mediated regression (catagen), rest (telogen), and shedding (exogen). Anagen is the growth phase of hair, during which the hair follicle is actively producing new hair cells. This phase lasts anywhere from two to six years, depending on individual genetics and other factors. Catagen is the transitional phase that occurs after the anagen phase. During catagen, the hair follicle shrinks and detaches from the blood supply, which eventually causes the hair shaft to break off from the follicle. Telogen is the resting phase of the hair growth cycle. During this phase, the hair follicle remains dormant for several months, and the old hair shaft remains attached to the scalp. Eventually, the old hair shaft will shed in exogen, and the growth cycle will begin again with the anagen phase. The lowest of three segments of the HHF, below the upper segment infundibulum and the mid-segment isthmus, is referred to as the bulge and contains arrector pili muscle and HHF stem cells (HHFSC) integral to follicle regeneration.

The speed of hair growth varies widely depending on individual genetics, age, and other factors. On average, human hair grows at a rate of approximately half an inch per month. However, some people may experience much slower or faster rates of hair growth. Hair growth can also be influenced by various external factors, such as nutrition, stress, and hormonal imbalances. Proper nutrition, including adequate protein and vitamin intake, is essential for healthy hair growth. Stress can cause hair loss or slow down the growth cycle, and hormonal imbalances can also affect hair growth and quality.

Hair loss is a common problem that affects millions of people worldwide. Hair loss can have a profound psychological impact on individuals, and many people who experience hair loss struggle with a decreased sense of self-worth, anxiety, and depression. Hair is often seen as a symbol of youth, vitality, and attractiveness, and losing it can cause feelings of shame, embarrassment, a sense of loss, and a significant source of stress and anxiety. It can lead to negative thoughts and feelings about one's appearance, and it can affect social interactions, personal relationships, and professional opportunities. Studies have shown that people with hair loss are more likely to experience anxiety and depression, and they may have lower self-esteem and poorer quality of life. Hair loss can be especially distressing for women, as they are often judged more harshly than men for their appearance. People with hair loss may feel self-conscious about their appearance, and they may avoid social situations or feel uncomfortable in public. Hair loss can have a significant impact on self-esteem and quality of life, and may be treated through a variety of methods, including medication, hair transplants, or lifestyle changes although current methods are not effective for most. Although such hair loss or thinning occurs in both men and women, it can be particularly distressing for women, as they are often judged more harshly than men for their appearance. Despite the significant psychological impact of hair loss, current treatments for this condition have not been entirely successful in providing a permanent solution. While treatments can stimulate hair growth and slow down hair loss, they do not work for everyone, and they have limitations and side effects.

In some individuals, hair loss or thinning and female pattern hair loss (FPHL) and male pattern hair loss (MPHL) or androgenetic alopecia (AGA) may occur due to a variety of factors. Disease, genetics, chemical exposure, nutrition, environmental or other stressors (lifestyle), hormonal imbalances, certain medical conditions, and the aging process itself can contribute to reduction in hair density and/or thickness of individual hairs, resulting in a hair loss and/or an overall thinning appearance of the hair. Alopecia areata (AA) is a form of non-cicatricial hair loss, that involves disruption of the HHF cycle, in which autoimmune generated follicular inflammation causes premature termination of the anagen phase. FPHL is a common form of nonscarring hair loss that primarily occurs in post-menopausal adult females. The condition is characterized by the progressive loss of terminal hairs over the frontal and vertex regions of the scalp, resulting in a visible reduction in hair density.

Some forms of human hair thinning (HHT) or FPHL may be due to a gradual change in the hair follicle immune privilege (IP) that is not caused by an autoimmune response that results in Alopecia areata but that can otherwise lead to hair loss or thinning, particularly age-related hair loss or thinning. Alopecia areata does involve immunomodulation of the IP but it is generated by antigens that are independent of age or aging and can occur in children and adults.

Immune privilege (IP) is a multifactorial process that limits recognition of foreign antigens, promotes immune tolerance (vs. immune response), and suppresses immune-mediated follicular microinflammation. The scientific literature describes one role of IP as protecting the bulge. S Azzawi, L R Peni and M M Senna, Skin Appendage Disord. 2018; 4:236-244.

There has been and remains a need for safe and effective topically applied compositions for treating hair loss or thinning that can increase both the number and diameter of terminal hairs. There is also a need for a safe and effective treatment composition and method that can treat and/or prevent age-related FPHL and MPHL or age-related hair thinning of adult males and females by use of topical compositions for application to the human scalp that help maintain or restore hair follicle IP. Those needs may be met by the embodiments of treatment compositions and methods of the present disclosure as further described below.

### SUMMARY OF THE DISCLOSURE

The present disclosure relates to compositions and methods that use one or more chlorin compounds and/or complexes, or other chlorophyll-related porphyrin compounds, for the treatment and/or prevention of aging hair loss or age-related hair thinning. In some embodiments, the use of such chlorin compounds and/or complexes may restore HHF immune privilege (IP) by treating and/or preventing human hair thinning (HHT) and/or FPHL and MPHL.

In some preferred embodiments, the chlorin compounds comprise one or more copper chlorin compounds or chelated copper chlorin compounds. In other preferred embodiments, the chlorin compounds comprise one or more copper chlorin complexes or chelated copper chlorin complexes. Unless one or the other is specifically being referenced, both copper chlorin compounds and copper chlorin complexes are referred to herein generally as copper chlorin for brevity and include acid and salt forms thereof unless specifically excluded.

In still other preferred embodiments, the chlorin compounds comprise a chelated zinc chlorin, a chelated iron chlorin, a chelated nickel chlorin, a chelated calcium chlorin, or a chelated manganese chlorin. Unless a specific divalent metal chelate is being referenced, references herein to a chlorin generally refer to any of a copper chlorin or any of the foregoing other metal chelated chlorins. Additionally, the terms “chelated[metal] chlorin” and “[metal] chlorin” (e.g., chelated copper chlorin and copper chlorin) are used interchangeably herein.

In some embodiments, a treatment composition comprises a safe and effective concentration of chlorin for treatment and/or prevention of one or more of hair loss or hair thinning conditions including HHT, AA, AGA, and FPHL. In some embodiments, a treatment composition comprises around 0.005% to about 0.2% of chlorin by weight. In more preferred embodiments, a treatment composition comprises around 0.01% to 0.1% of chlorin by weight. In a most preferred embodiment, a treatment composition comprises around 0.025% to 0.1% of chlorin by weight.

In some embodiments, a treatment composition comprises a safe and effective concentration of copper chlorin for treatment of hair loss or thinning. In some embodiments, a treatment composition comprises around 0.025% to about 0.1% of copper chlorin by weight.

In some embodiments, a copper chlorin used in a treatment composition herein comprises at least 75% by weight of one or more of trisodium copper (II) chlorin e6, disodium copper (II) isochlorin e4, sodium copper (II) rhodin g7, sodium copper (II) rhodochlorin, or sodium copper (II) chlorin p6.

In still other embodiments, a copper chlorin used in a treatment composition herein comprises (1) at least 30% by weight (total) of (a) disodium copper (II) isochlorin e4 or (b) trisodium copper (II) chlorin e6 or (c) a combination thereof and (2) one or more of (a) chlorin e4, (b) isochlorin e4, (c) copper chlorin e4, (d) copper isochlorin e4, (e) chlorin e6, (f) copper chlorin p6, (g) copper pheophorbide a, (h) copper pyropheophorbide a, (i) copper purpurin 7, (j) copper rhodin g7, (k) copper rhodochlorin, and (1) oxidized forms and/or salts of (a)-(k).

In other embodiments a copper chlorin used in a treatment composition herein comprises an acid or salt forms of (a) copper (II) chlorin e6, (b) copper (II) isochlorin e4, (c) copper (II) chlorin p6, (d) copper (II) rhodin g7, (e) copper (II) rhodochlorin, (f) copper (II) purpurin 7, and/or (g) copper (II) chlorin e4 wherein the valence of the chelated copper compound of (a)-(g) is +2 expressed as (II).

In another preferred embodiment, a copper chlorin used in a treatment composition disclosed herein is part of a copper chlorophyllin complex salt. In some embodiments, a copper chlorophyllin complex salt is a sodium, potassium, or sodium potassium salt or mixture thereof. In these embodiments, a copper chlorin from the copper chlorophyllin complex salt may comprise an acid or salt form of one or more of (a) copper (II) chlorin e6, (b) copper (II) isochlorin e4, (c) copper (II) chlorin p6, (d) copper (II) rhodin g7, (e) copper (II) rhodochlorin, (f) copper (II) purpurin 7, and (g) copper (II) chlorin e4. The chlorophyllin copper complex may also be called chlorophyllin cu complex, chlorophyllin copper sodium complex, chlorophyllin-copper complex, copper sodium chlorophyllin, copper sodium complex chlorophyllin, or simply copper chlorophyllin and will also be referred to in this application as a copper chlorin complex.

In another embodiment, a treatment composition comprises alkali salts of copper chlorins such as, for example, sodium and potassium salts which may be mono-, di-, or tri-alkali salts or a combination of sodium and potassium salts of the chelated copper chlorin compounds.

In another embodiment, a treatment composition comprises acid or salt forms of copper chlorin wherein the copper (II) metal chelate is replaced with other divalent metal chelates that may include one or more of zinc, iron, nickel, calcium, and manganese.

In some embodiments, a treatment composition contains one or more prodrugs of a copper chlorin compound wherein the carboxylic acid groups emanating from one or more of the number 13, 15, and 17 positions of the tetrapyrrole core is fully or partially esterified by one or more alkyl, acyl, or fatty ester groups and wherein the resulting copper chlorin compound or salt thereof is, in the case of ester prodrug modification, a mono-ester, di-ester, or tri-ester or a mixture thereof and wherein the numbering of the tetrapyrrole core of the copper chlorin follows that described by Tumolo, T., & Lanfer-Marquez, U. M. (2012). Copper chlorophyllin: A food colorant with bioactive properties?. Food Research International, 46(2), 451-459, which is incorporated herein by reference and the numbering system as used in the Tumolo reference is included as FIG. 3 wherein the 13, 15, and 17 carbon positions of the tetrapyrrole group of various chlorin compounds are clearly labeled. For example, a prodrug may be a monoethyl, diethyl, or triethyl ester or a combination thereof. In other embodiments, a prodrug ester group may be supplied by C2-alkoxy or longer chain group or by short chain glycols or by polyethylene or polypropylene glycols. In other embodiments one or more of the carboxyl groups in the 13, 15, and 17 positions may be modified to create phosphate, carbamate, amide, or carbonate copper chlorin compounds. Such prodrug examples can improve the topical skin penetration, stability, and solubility of the parent copper chlorin compound and may also be used to change the pharmacodynamics and pharmacokinetics of the parent, unmodified copper chlorin compound to increase the tissue residence time and prolong the biological activity.

In some embodiments, a treatment composition is topically applied. In some embodiments, a topical treatment composition comprises a copper chlorin and a carrier vehicle suited for topical application of a treatment composition to human skin, particularly a human scalp. A carrier vehicle may be aqueous or non-aqueous wherein the vehicle is in the form of a solution, suspension, gel, emulsion, microemulsion, nanosuspension, liposomal dispersion, lotion, cream, solid, aerosol or non-aerosol spray. When a non-aqueous pharmaceutical vehicle, or a solid pharmaceutical vehicle, or an aqueous gel, lotion, or cream pharmaceutical vehicle is used, copper chlorin is preferably evenly dispersed or solubilized therein.

In some embodiments, a treatment composition comprises a copper chlorin solubilized in a topical aqueous solution or gel or solubilized in the water phase of an emulsion or microemulsion.

In other embodiments, acid or non-salt forms, prodrug forms or other non-water soluble forms of copper chlorins are first dissolved in pharmaceutically or cosmeutically acceptable solvents and further incorporated in the non-aqueous phase of an emulsion, microemulsion, nanoemulsion, or anhydrous vehicle.

In other embodiments, a treatment composition comprises copper chlorin in a pharmaceutical vehicle comprising one or more pharmaceutical grade compounds. Such pharmaceutical grade compounds may include aqueous solvents, non-aqueous solvents, skin penetrants or penetration enhancing agents, antioxidants, preservatives, emulsifiers, viscosity modifiers, thickeners, surfactants, emollients, humectants, keratolytics, skin softening agents, emulsion stabilizers, pH adjusters, buffering agents, suspending agents, UV-A filters, UV-B filters, UV-C-filters, mineral oils, mineral waxes, vegetable oils, vegetable waxes, polyethylene glycols, PEG glyceryl esters, film-forming polymers, emollients, and/or opacifiers. Any of these pharmaceutical grade compounds may also be excluded from a treatment composition according to some embodiments.

In other embodiments, a treatment composition may be enhanced by encapsulating the treatment composition by creating a liposomal, nanosomal, nanoparticle or niosomal skin delivery vehicle wherein the liposomal delivery system is one or more of small unilamellar vesicles, large unilamellar vesicles, multilamellar vesicles, and multivesicular vesicles.

In these embodiments, a treatment composition may comprise one or more other active ingredients, which may include pharmaceutical grade compounds as previously described, that aid in topical skin penetration, tissue residence time, stability, and/or solubility of the copper chlorin. In these embodiments, a treatment composition may also include one or more other inactive ingredients, vehicle ingredients, and/or pharmaceutical grade compounds.

In some embodiments, copper chlorin is the only active ingredient in a treatment composition for treatment of hair loss or thinning. In these embodiments, a treatment composition may comprise one or more inactive ingredients and/or vehicle ingredients.

In other embodiments, the treatment composition may be enhanced with the direct addition to compositions of the embodiment or separately dosed oral or topical application of compositions containing one or more ingredient that includes ashwagandha, marine collagen, caffeine, tocotrienol, curcumin, saw palmetto, kelp, resveratrol, horsetail, keratin, amino acids, black pepper, capsicum, vitamin A, vitamin C, vitamin D, biotin, zinc, selenium, hyaluronic acid, sodium hyaluronate, burdock root extract, marigold extract, apple extract, sandalwood extract, bovine milk extract. milk peptides and proteins, calcium salts, shark cartilage, oyster powder extract, L-cystine, L-methionine, essential oils including one or more of thyme, rosemary, lavender, and cedarwood oils, green tea, pumpkin seed extract, carrot extract, tomato extract, quercetin, coenzyme q10, vitamin k2, garlic extract. and extract of blue-green algae. In other embodiments, one or more of these ingredients may be excluded from treatment compositions and/or methods of treating, such as separate oral doses or topical applications.

In still other embodiments, the treatment composition may contain or be supplemented with oral, topical or injectable dosages of minoxidil, duasteride, spironolactone, finasteride, one or more 5-alpha reductase inhibitors, Janus kinase (JAK) inhibitors, baricitinib, metformin, cyclosporin A, hydrocortisone, desonide, fluocinolone, clobetasol, 2,2,6,6-tetramethyl-1-piperidinyloxy (TEMPO), 4-hydoxy-TEMPO, hydroxychloroquine, triamcinolone acetonide, platelet-rich plasma (PRP), and sulfasalazine. In other embodiments, one or more of these ingredients or compositions may be excluded from treatment compositions and/or methods of treating, such as separate oral doses, topical applications, and/or injections.

In other embodiments, the treatment composition may be combined with or supplement by medical device treatments that use one or more treatments with topical cryotherapy using liquid carbon dioxide, low level laser light with red or blue light, topical ionized nitrogen gas treatment such as Neogen Plasma, and microneedling.

Methods for treating and/or preventing hair loss or thinning, or methods for increasing a density and/or diameter of human hair, according to embodiments herein comprise applying to any skin surface area of the human body displaying a loss, reduction or thinning of terminal hair growth, a topical composition (also referred to herein as a topical hair volumizing composition) comprising a chlorin. In some embodiments, a method comprises applying a topical composition comprising a copper chlorin to any area of skin surface of the human body displaying a loss, reduction or thinning of terminal hair growth. In some embodiments, a topical composition applied is a treatment composition according to an embodiment herein. In some embodiments, a treatment composition used in a method of treating and/or preventing hair loss or thinning is comprised of, consists essentially of, or consists of a chelated copper chlorin compound, wherein the chelated copper chlorin compound is comprised of at least 75% of a chlorin species selected from the group consisting of (a) trisodium copper (II) chlorin e6 and (b) disodium copper (II) isochlorin e4, and the chelated copper chlorin compound is present at a concentration of from 0.02 to 0.1% by weight of the composition.

In certain embodiments, disodium copper (II) isochlorin e4 comprises at least 30% by weight of the chelated copper chlorin compound, and the chelated copper chlorin is further comprised of at least one additional chlorin compound selected from: chlorin e4; isochlorin e4; copper chlorin e4; copper isochlorin e4; chlorin e6; copper chlorin p6; copper pheophorbide a; copper pyropheophorbide a; copper purpurin 7; copper rhodin g7; copper rhodochlorin; and oxidized forms and salts of the above listed chlorin compounds.

Use of treatment compositions and/or methods according to some embodiments herein may aid in reducing follicle microinflammation that occurs due to age-related improvement in the IP and/or improvement in the anagen phase growth cycle. The improvement in the anagen growth cycle mediated by application of use of the treatment composition of the embodiment can result based on a change in the follicle immune response and improvement in IP. The improvement in hair thickness and density after topical treatment by embodiments herein is further characterized by the downregulation of the expression of certain antibody genes. Genes that may be downregulated by a treatment composition according to some embodiments comprise genes that code for: human immunoglobulin kappa light chain variable region (IGVK) protein(s), human immunoglobulin lambda variable light chain (IGVL) protein(s), human immuno-globulin heavy chain (IgH) protein(s), or a combination thereof. Preferably, treatment compositions herein may downregulate these genes by at least 200%. Specific genes that may be downregulated by a treatment composition according to some embodiments comprise one or more of IGKV1 D-16; IGKV3-20; IGKV3-11; IGKV2D-28; IGKV2D-28; IGLV3-19; IGLV1-40; IGLV1-51; IGLV2-11; IGLV1-51; IGHV3-48; IGHV3-30; IGHV3-53; IGHV3-7; PRSS21; GZMK; ICOS; CXCL13; CXCL10; and/or CCL24. A minority of immunoregulatory genes may also be upregulated. Specific genes that may be upregulated by a treatment composition according to some embodiments comprise genes that code for: IGLV2-8 and/or IGLV2-23. In some embodiments, treatment compositions and/or methods herein upregulate these genes by at least 200%.

In some embodiments, a dose of a treatment composition comprising a chlorin is applied once to three times a day on a daily, once weekly, twice weekly, three times weekly, or on every other day basis over a treatment period. The treatment period may be anywhere from 12 weeks or longer. A dose of a treatment composition used in some method embodiments herein may be around 0.5-2 mL or 0.5-2 g per around 700 cm² of a scalp surface (or other skin surface) area being treated. Such a dose of a treatment composition may comprise around 3-13 μg of chlorin per cm² of skin surface area.

In other embodiments, a dose of a treatment composition comprising a chlorin is applied topically at least once daily, for at least 3 months, preferably at least 6 months, to the scalp of a person having age-related HHT, AGA, FPHL or MPHL.

In some embodiments, a treatment composition is a leave-on composition that is applied to a hair area after shampooing (or wetting) the hair (if the hair is shampooed or wetted on a day of treatment composition application) and remains on the scalp/hair/hair area for at least 6 hours, more preferably at least 8 hours prior to the hair area being shampooed or wetted. Multiple applications of a treatment composition may be applied over days within a treatment period without shampooing or wetting the hair area, but if it is shampooed or wetted, it is preferably not done until a treatment composition has been in contact with the hair area for at least 6 hours.

Use of treatment compositions and/or methods herein may provide advantages in increasing density and/or thickness of hair in a hair area that is treated compared to the same hair area prior to treatment. Treatment compositions and/or methods herein may achieve (1) an increase of average density of terminal hairs of greater than 15%, more preferably greater than 25%, and/or (2) an increase of average diameter of terminal hairs of greater than 5%, more preferably greater than 15%. Treatment compositions according to embodiments herein may also reduce the loss of pigment in human hairs that result in graying hair. Treatment compositions and/or methods herein may achieve a reduction in vellus or non-terminal hair density by greater than 25%, preferably by 50%, and most preferably by greater than 75%.

### BRIEF DESCRIPTION OF THE DRAWINGS

The compositions and methods of the disclosure are further described and explained in relation to the following figures wherein:

FIGS. 1a and 1b are color photographs of Subject 001 at baseline and 90±10 days after once-daily application of a topical hair volumizing composition containing a chelated copper chlorin compound in accordance with methods of the present invention;

FIGS. 2a and 2b are color photographs of Subject 006 at baseline and 90±10 days after once-daily application of a topical hair volumizing composition containing a chelated copper chlorin compound in accordance with methods of the present invention;

FIG. 3 shows the numbering system for the 13, 15, & 17 ring positions and potential reaction sites of the hydroxyl groups emanating from these ring positions of the tetrapyrrole structures of selected chlorin compounds as published by Tumolo et al in Food Research International 46 (2012) 451-459;

FIG. 4 is a schematic showing a stepwise procedure for production of RNA using RNeasy Plus procedure;

FIG. 5 is a graph showing Gene Ontology terms most enriched in cell populations treated with trisodium copper (II) chlorin e6; and

FIG. 6 is a graph showing Gene Ontology terms most enriched in cell populations treated with diethyl ester of copper (II) chlorin e6.

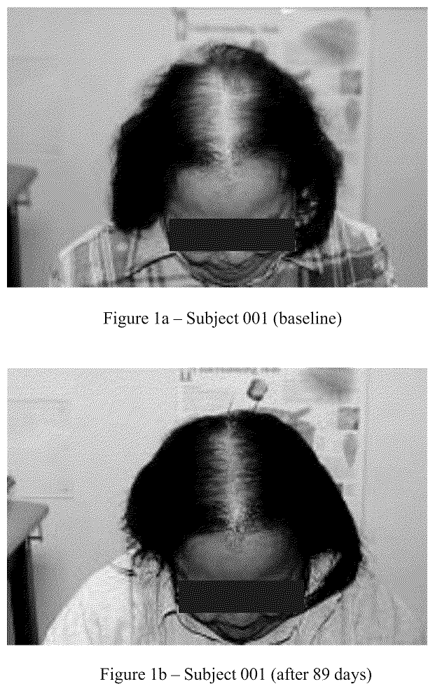

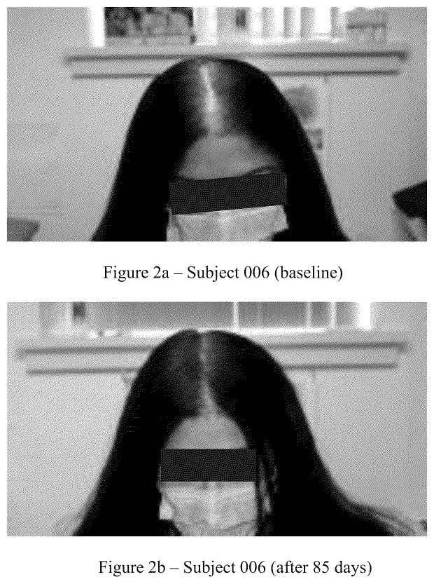

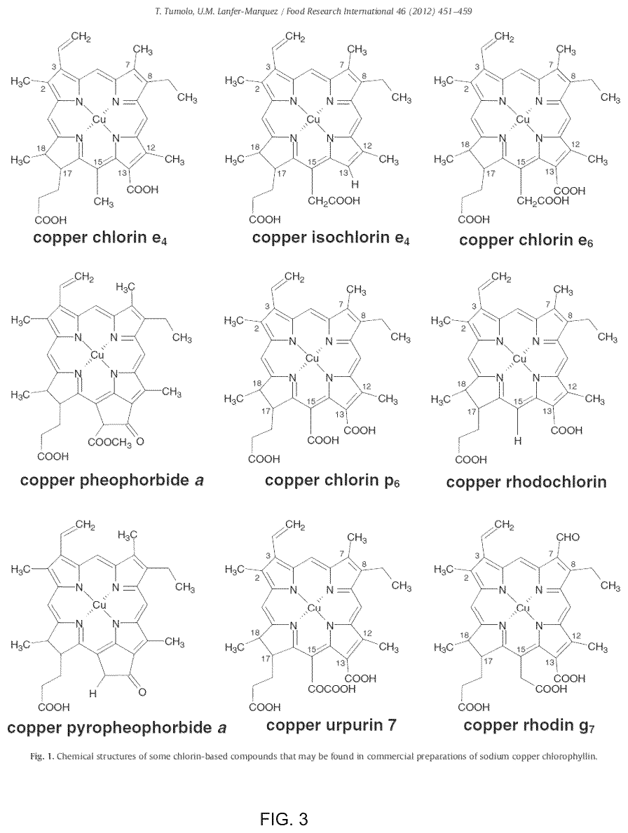

### DETAILED DESCRIPTION

The present disclosure relates to compositions and methods that use one or more chlorins or other chlorophyll-related porphyrin compounds for the treatment and/or prevention of hair loss or thinning. Application of treatment compositions according to embodiments herein may treat and/or prevent age-related HHT. Application of treatment compositions according to embodiments herein may also restore age-related HHF IP.

In some embodiments, application of treatment compositions according to embodiments herein may increase the density and/or thickness of human terminal hairs and reduce the density of human vellus hairs. “Terminal hair” has a diameter of 40 microns or greater. “Vellus hair” has a diameter of less than 40 microns. Vellus hair is thin, fine body or facial hair that generally has no pigment. References herein to vellus hair density refer to the density of vellus hairs, by number of total, individual hairs, in a defined surface area. References herein to an average vellus hair density refers to an average number of the total, individual vellus hairs in a larger defined surface area based on the number of total vellus individual hairs in one or more smaller surface areas. References herein to terminal hair density refers to (1) the density of terminal hairs by number of total, individual hairs, in a defined surface area and/or (2) an average density of terminal hairs in the defined surface area. References herein to an average terminal hair diameter refers to (1) an average diameter of the total, individual terminal hairs in a larger defined surface area based on the diameters of total individual terminal hairs in one or more smaller surface areas and/or (2) an average diameter of terminal hairs in the larger defined surface area based on the average diameter of terminal hairs in the one or more smaller surface areas. References herein to hair thickness refer to a diameter of an individual terminal hair. References herein to an average hair thickness or average diameter of terminal hairs refer to an average of the diameters of individual terminal hairs in a defined surface area on the skin or scalp. Terminal hairs are classified by different thicknesses: “thin” terminal hairs are between 40-80 microns; “normal” terminal hairs are between 80-110 microns; and “thick” terminal hairs are >110 microns.

A skin area of the scalp or face covered with predominately terminal hairs (including eyebrows, eyelashes, mustache, or beard) meeting one or more of the following criteria based on trichometric analysis of a measured surface area is considered thinning or as hair loss (collectively referred to as human hair thinning or HHT) for purposes of this disclosure if: (i) the average (mean) hair diameter of at least 25% of terminal hairs in the measured area is less than 80 microns; or (ii) the density of vellus hairs in the measured area is at least 15%; or (iii) the ratio of terminal hairs to vellus hairs in the measured area is about 5:1 or less; or (iv) one standard deviation of the mean diameter of terminal hairs divided by the mean diameter of terminal hairs is 0.2 or greater; or (v) any combination thereof. A typical surface area measured to determine if hair is considered thinning is around 0.7 to 0.9 cm², such as around 83.2 mm².

Thinning hair may be visible and appear as a receding hairline and/or progressive U-shaped bald spots in men and thinning hair along the crown or midline of the scalp in women. Thinning hair may, however, not be visible, but can be seen under magnification (hi-resolution photography, trichoscopy, or microscope). Hair may also be characterized as age-related HHT for purposes of this disclosure based on one or more of the following observations: (i) perceived widening of the hair part; (ii) seeing more skin on the scalp (e.g., when hair is pulled back); (iii) more hair on a pillow after a sleeping on the pillow; (iv) more hair strands on a brush; more hair in the drain; (v) sunburn on the scalp; (vi) hair pull test (six or more stands fall out when about 40 strands are grasped from different parts of the scalp and gently tugged); (vii) hair tug test (hair breaks when a section of hair is grasped with two hands, one near the root and one near the tip, and tugged); or (viii) a thinner ponytail or increased looping of the holder around the ponytail.

### Treatment Compositions

In some embodiments, topical hair volumizing compositions for treating or preventing age-related hair loss or hair thinning comprise a chlorin, preferably at least one copper chlorin. In some embodiments, a topical hair volumizing composition comprises one or more active ingredients useful in increasing the density and/or the diameter of terminal hairs and a carrier. In some embodiments, the one or more active ingredients comprises one or more chlorins. In other embodiments, the one or more active ingredients consists of one or more chlorins. In still other embodiments, the one or more active ingredients comprises one or more chelated copper chlorin compounds. In still other embodiments, the one or more active ingredients consists of one or more chelated copper chlorin compounds.

Preferred chlorins for use in a treatment composition herein are copper chlorins, zinc chlorins, iron chlorins, nickel chlorins, calcium chlorins, and manganese chlorins. Any of these chlorins may be used individually to the exclusion of any other chelated metal chlorin, such as the use of only one or more copper chlorins without any iron chlorins, nickel chlorins, calcium chlorins, magnesium or manganese chlorins. Any of these chlorins may also be used in any combination with one or more other chelated metal chlorins, such as the use of one or copper chlorin with one or more iron chlorins.

In some embodiments, a treatment composition may comprise one or more chlorins in a total concentration of around 0.001% to about 0.5%, more preferably around 0.01% to 0.25%, and most preferably around 0.02% to 0.1% by weight of the treatment composition. In some embodiments, a treatment composition may comprise one or more copper chlorins in a total concentration of around 0.001% to about 0.5%, more preferably around 0.01% to 0.25%, and most preferably around 0.02% to 0.1% by weight of the treatment composition. The balance of the treatment composition in these embodiments may comprise a carrier (also referred to herein as a vehicle or a pharmaceutical carrier or a pharmaceutical vehicle) with or without one or more other active ingredients and with or without one or more inactive ingredients. Other active ingredients may comprise ingredients that enhance functionality of the copper chlorin. Such enhanced functionality may include aiding in topical skin penetration, tissue residence time, stability, and/or solubility of the copper chlorin and/or other hair growth ingredient(s).

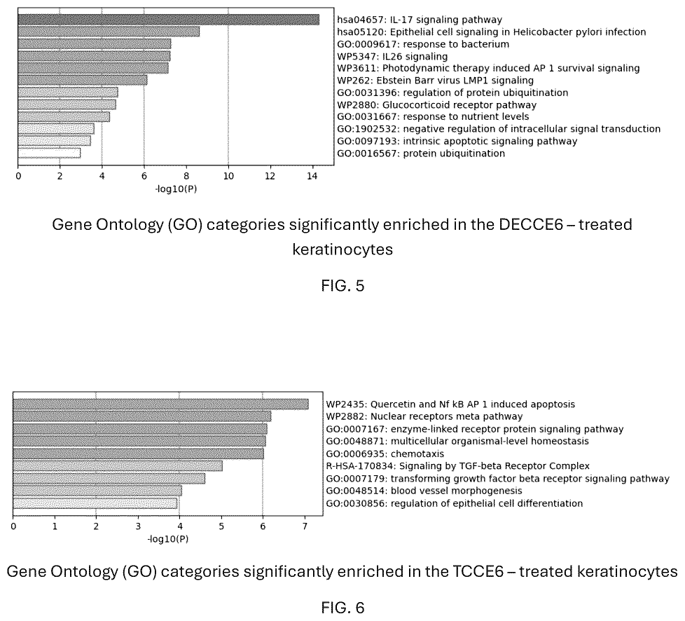

A treatment composition may comprise one or more of: (1) trisodium copper (II) chlorin e6, (2) disodium copper (II) isochlorin e4, (3) sodium copper (II) rhodin g7, (4) sodium copper (II) rhodochlorin, (5) sodium copper (II) chlorin p6, (6) chlorin e4, (7) isochlorin e4, (8) copper chlorin e4, (9) copper isochlorin e4, (10) chlorin e6, (11) copper chlorin p6, (12) copper pheophorbide a, (13) copper pyropheophorbide a, (14) copper purpurin 7, (15) copper rhodin g7, (16) copper rhodochlorin, (17) copper chlorophyllin complex salt (preferably a sodium, potassium, or sodium potassium salt or mixture thereof), (18) acid or salt forms of copper chlorin wherein the copper (II) metal chelate is replaced with other divalent metal chelates that may include one or more of (a) zinc, (b) iron, (c) nickel, (d) calcium, (e) magnesium and (f) manganese, (19) alkali salts of copper chlorins such as, for example, sodium and potassium salts which may be mono-, di-, or tri-alkali salts or a combination of sodium and potassium salts of the chelated copper chlorin compounds, (20) one or more prodrugs of a copper chlorin compound wherein the carboxylic acid groups emanating from one or more of the number 13, 15, and 17 positions of the tetrapyrrole core is fully or partially esterified or modified by one or more C-2 or greater alkoxy ester, carbamide, phosphate, amide or other prodrug modifications of the chlorin tetrapyrrole hydroxyl or carboxylic acid groups and wherein the resulting copper chlorin compound or salt thereof is a mono-ester, di-ester, or tri-ester or a mixture thereof or other prodrug modification of the hydroxyl and carboxylic acid groups emanating from the 13, 15, and 17 positions of the tetraprryole core of the copper chlorin and wherein the numbering of the tetrapyrrole core of the copper chlorin follows that described by Tumolo, T., & Lanfer-Marquez, U. M. (2012). Copper chlorophyllin: A food colorant with bioactive properties?. Food Research International, 46(2), 451-459, as shown in FIG. 5.

Disodium copper (II) isochlorin e4 is a porphyrin compound also known as disodium 3-[(2Z,4S,5S,16Z)-2-(carboxylatomethyl)-10-ethenyl-15-ethyl-5,9,14,19-tetramethyl-21,23,24,25-tetraaza-22-cuprahexacyclo[9.9.3.1\hat{ }{3,6}.1\hat{ }{13,16}.0\hat{ }{8,23}.0\hat{ }{18,21}]pentacosa-1(20),2,6(25),7,9,11,13(24),14,16,18-decaen-4-yl]propanoate. Neither a CAS number nor an EINECS number has been assigned to this compound. Trisodium copper (II) chlorin e6 is a porphyrin compound and has been assigned CAS number 11006-34-1 and EINECS number 234-242-5.

A copper chlorin used in a treatment composition may comprise at least 75% by total weight of one or more of (1) trisodium copper (II) chlorin e6, (2) disodium copper (II) isochlorin e4, (3) sodium copper (II) rhodin g7, (4) sodium copper (II) rhodochlorin, or (5) sodium copper (II) chlorin p6. A copper chlorin used in a treatment composition may comprise at least 80%, preferably at least 85%, more preferably at least 90%, and most preferably at least 95% by total weight of one or more of the specific copper chlorins in (1)-(5) in this paragraph.

A copper chlorin used in a treatment composition may comprise at least 75% by weight, or at least 80% by weight, preferably at least 85% by weight, more preferably at least 90% by weight, and most preferably at least 95% by weight of trisodium copper (II) chlorin e6. A copper chlorin used in a treatment composition may comprise at least 75% by weight, or at least 80% by weight, preferably at least 85% by weight, more preferably at least 90% by weight, and most preferably at least 95% by weight of disodium copper (II) isochlorin e4. A copper chlorin used in a treatment composition may comprise at least 75% by weight, or at least 80% by weight, preferably at least 85% by weight, more preferably at least 90% by weight, and most preferably at least 95% by weight of sodium copper (II) rhodin g7. A copper chlorin used in a treatment composition may comprise at least 75% by weight, or at least 80% by weight, preferably at least 85% by weight, more preferably at least 90% by weight, and most preferably at least 95% by weight of sodium copper (II) rhodochlorin. A copper chlorin used in a treatment composition may comprise at least 75% by weight, or at least 80% by weight, preferably at least 85% by weight, more preferably at least 90% by weight, and most preferably at least 95% by weight of sodium copper (II) chlorin p6.

A treatment composition herein may also comprise other porphyrin compounds, other specific copper chlorins, and/or specific combinations thereof. In some embodiments, a treatment composition comprises (1) a chlorin that is at least 30% by weight (total, of the chlorin, not the treatment composition) of (a) disodium copper (II) isochlorin e4 or (b) trisodium copper (II) chlorin e6 or (c) a combination thereof or (d) oxidized forms and/or salts of (1)(a)-(1)(c) and (2) one or more of (a) chlorin e4, (b) isochlorin e4, (c) copper chlorin e4, (d) copper isochlorin e4, (e) chlorin e6, (f) copper chlorin p6, (g) copper pheophorbide a, (h) copper pyropheophorbide a, (i) copper purpurin 7, (j) copper rhodin g7, (k) copper rhodochlorin, and (I) oxidized forms and/or salts of (2)(a)-(2)(k).

In other embodiments, a treatment composition herein comprises (1) a chlorin that is at least 30% by weight (total, of the chlorin, not the treatment composition) of (a) disodium copper (II) isochlorin e4 or (b) trisodium copper (II) chlorin e6 or (c) a combination thereof or (d) oxidized forms and/or salts of (1)(a)-(1)(c) and (2) at least one chlorin compound selected from the group consisting of (a) chlorin e4, (b) isochlorin e4, (c) copper chlorin e4, (d) copper isochlorin e4, (e) chlorin e6, (f) copper chlorin p6, (g) copper pheophorbide a, (h) copper pyropheophorbide a, (i) copper purpurin 7, (j) copper rhodin g7, (k) copper rhodochlorin, and (1) oxidized forms and/or salts of (2)(a)-(2)(k). In other embodiments a treatment composition herein comprises an acid or salt forms of (1) copper (II) chlorin e6; (2) copper (II) isochlorin e4; (3) copper (II) chlorin p6; (4) copper (II) rhodin g7; (5) copper (II) rhodochlorin; (6) copper (II) purpurin 7; and/or (7) copper (II) chlorin e4 wherein the valence of the chelated copper is +2 expressed as (II) in compounds (1)-(7).

In other embodiments, a treatment composition comprises a chelated copper chlorin compound that is comprised of at least 75%, preferably at least about 80%, more preferably at least about 85%, still more preferably at least about 90%, even more preferably at least about 95% of disodium copper (II) isochlorin e4. In these compositions, disodium copper (II) isochlorin e4 is present at a concentration of from about 0.001% to about 0.5%, preferably from about 0.01% to 0.25%, and more preferably from about 0.02% to about 0.1%.

In still other embodiments, topical hair volumizing compositions used in practicing the methods of the present invention contain a chelated copper chlorin compound that is comprised of at least 75%, preferably at least about 80%, more preferably at least about 85%, still more preferably at least about 90%, even more preferably at least about 95% of trisodium copper (II) chlorin e6. In these compositions, trisodium copper (II) chlorin e6 is present at a concentration of from about 0.001% to about 0.5%, preferably from about 0.01% to 0.25%, and more preferably from about 0.02% to about 0.1%.

In other embodiments, topical hair volumizing compositions used in practicing the methods of the present invention contain a chelated copper chlorin compound comprised of (a) at least 30% by weight of disodium copper (II) isochlorin e4 or trisodium copper (II) chlorin e6 and (b) at least one chlorin compound selected from the group consisting of chlorin e4; isochlorin e4; copper chlorin e4; copper isochlorin e4; chlorin e6; copper chlorin p6; copper pheophorbide a; copper pyropheophorbide a; copper purpurin 7; copper rhodin g7; copper rhodochlorin; and oxidized forms and salts of the above listed chlorin compounds. In these embodiments, the chelated copper chlorin compound is present in the topical hair volumizing composition at a concentration of from about 0.001% to about 0.5%, preferably from about 0.01% to 0.25%, and more preferably from about 0.02% to about 0.1%.

In still other embodiments, the topical hair volumizing compositions used in practicing the methods of the present invention contain two chelated copper chlorin compounds: a first chelated copper chlorin compound comprised at least 75% by weight disodium copper (II) isochlorin e4 or trisodium copper (II) chlorin e6; and a second chelated copper chlorin compound comprised-(a) at least 30% by weight of disodium copper (II) isochlorin e4 and (b) at least one chlorin compound selected from the group consisting of chlorin e4; isochlorin e4; copper chlorin e4; copper isochlorin e4; chlorin e6; copper chlorin p6; copper pheophorbide a; copper pyropheophorbide a; copper purpurin 7; copper rhodin g7; copper rhodochlorin; and oxidized forms and salts of the above listed chlorin compounds. In these embodiments, the first and second chelated copper chlorin compounds are present in the topical hair volumizing composition at a combined concentration of from about 0.001% to about 0.5%, preferably from about 0.01% to 0.25%, and more preferably from about 0.02% to about 0.1%.

Topical hair volumizing compositions according to some embodiments herein may contain a carrier, or vehicle (also referred to as a carrier vehicle) which are known to the person having ordinary skill in the art. In some embodiments, a treatment composition comprises a copper chlorin and a carrier vehicle. A carrier vehicle may be one suited for topical application of a treatment composition. A carrier vehicle may be an aqueous or in a non-aqueous pharmaceutical vehicle in which a chlorin is miscible or dispersible and can be administered in different forms, including an aerosol spray, non-aerosol spray, foam, cream, lotion, gel, serum, solution, suspension, emulsion, microemulsion, nanosuspension, liposomal dispersion, or solid. One preferred dosage form for a treatment composition according to some embodiments is a hydro-alcoholic spray administered through a trigger or pump sprayer, a pressurized aerosol sprayer, or a bag-on-valve sprayer. When a non-aqueous pharmaceutical vehicle, or a solid pharmaceutical vehicle, or an aqueous gel, lotion, or cream pharmaceutical vehicle is used, a copper chlorin is preferably evenly dispersed therein. A treatment composition according to embodiments herein may comprise water as part of a carrier, such as a water-in-oil emulsion, an oil-in-water emulsion, a water-in-silicone emulsion, a silicone-in-water emulsion, a water-in-oil-in-water emulsion, or a thickened aqueous gel. In some embodiments, a treatment composition may be anhydrous. Some carriers may include alcohol, glycols, esters, or silicones.

Topical hair volumizing compositions according to some embodiments may further comprise one or more ingredients that provide one or more functional or cosmetic benefit to skin or hair such as reducing one or more of dryness, oiliness, visible redness, hair strand brittleness or breakage; provide hydration, skin barrier and hair cuticle protection, and/or protection from ultraviolet radiation or other environmental stressors; impart shine or softness; improve combability; increase color vibrancy. Non-limiting examples of skin or hair benefit ingredients include one or more of antioxidants, anti-inflammatory agents, humectants, skin penetration enhancers, over-the-counter or prescription active pharmaceutical ingredients (including skin protectant drug products, anti-inflammatory, antimicrobial, antifungal, anti-dandruff, antipruritic, hair growth stimulant drug products, sunscreens), temporary, intermediate and permanent hair coloring systems, hair gloss agents, hair conditioning agents, botanical or marine extracts, growth factors, vitamins, UV protectants, peptides, and amino acids. Preferred antioxidants comprise tocopherols, tocotrienols, sulfite and bisulfite salts, BHA, BHT, retinoid esters, alcohols and aldehydes, superoxide dismutase, cysteine, N-acetyl cysteine, ubidecarenone, ubidecarenol, glutathione, ascorbic acid or sodium, calcium or potassium ascorbate, tetrahexadecyl ascorbate, ascobyl phosphate salts, ascorbyl palmitate, hydroxyacetophenone, Trolox®, resveratrol, cysteamine HCL, tert-butylhydroxyquinone, Tris(2,4-di-tert-butylphenyl)phosphite, hydroxytyrosol, epigallocatechin, ergothionine, alpha-arbutin, octadecyl-di-t-butyl-4-hydroxycinnamate, sodium pyruvate, ferulic acid and ferulate salts, gallic acid, propyl gallate, lutein, bakuchiol, citric acid and citrate salts, 1-methionine, 1-malic acid and malate salts. Preferred anti-inflammatories comprise niacinamide, glycyrrizin salts and licorice extracts, colloidal oatmeal, oatmeal beta glucans,_beta glucan, green tea extract, white tea extract, bisabolol, allantoin, superoxide dismutase, Phytoplenolin®, zinc salts, witch hazel extract, shea nut extract, one or more extracts of lavender, grapefruit, rosemary, oregano, sage, barley, feverfew, turmeric, Centella asiatica, cucumber, aloe vera, Peregrina seed, tamarindua Indica seed and stevioside, Avena sativa kernel, Sephora augustifolia root, Passiflora edulis fruit, Rosa damascana flower, Chrysanthenum indicum flower, Ocimum santum leaf, Phellodendron chinese root, Safflower seed extract and Cannabis sativa seed oil and phytosterols. Preferred skin protectant and moisturizing ingredients comprise sodium hyaluronate, hyaluronic acid, glycerin, propylene glycol, butylene glycol, pentylene glycol, hexylene glycol, polyethylene glycol, polypropylene glycol, water soluble or water-dispersible dimethicone or polyhexasiloxane, Biosaccharide Gum-4, Hydroxyethyl Methylthiopropenamide, water-soluble extracts of one or more of Myrciaria Dubia Fruit (and) Aureobasidium Pullulans Ferment, Crocus Sativus Flowers, Opuntia Ficus-Indica Flowrs Caesalpinia Spinosa Fruits, Melissa Officinalis Leaf; alpha-glucan oligosaccharides, methoxyphenylimino dimethylcyclohexenyl ethyl glycinate, resveratrol, acetyl hexapeptide-7, acetyl tetrapeptide-3, sh-polypeptide-123, soluble collagen, propanediol benzoate, milk protein, sh-oligopeptide-1, glucomannan diglycerol, trehalose, sorbitol, triethylene glycol, mannitol, sorbitol, acetamide MEA, sodium lactate, pyrrolidone carboxylic acid, urocanic acid, inulin, saccharide isomerate, glycogen, algae polysaccharides, chitin and chitosan polysaccharides, xanthum gum, algin, glycoproteins such as Immucell® and Revitalin® BT (DSM, Maastricht, Netherlands) and Bergenia glycoprotein, Betula glycoprotein, Chaga glycoprotein, Dendropanax glycoprotein, and Ganoderma glycoprotein (Durae Corp., Anyang-si, Gyeonggi-do, South Korea). Preferred antimicrobials and preservatives comprise phenoxyethanol, phenylethyl alcohol, benzoic acid and benzoate salts, sorbic acid and sorbate salts, ethylhexylglycerin, chloroxylenol, phenylpropanol, propanediol, gluconolactone, caprylyl glycol, pentylene glycol, o-Cymen-5-ol, 1. 2-hexanediol, and benzyl alcohol. Preferred anti-dandruff ingredients comprise Octopirox® [1-Hydroxy-4-methyl-6-(2,4,4-trimethylpentyl)pyridin-2(1H)-one], zinc pyrithione, Ciclopirox Olamine, Piroctone Olamine, micronized sulfur, selenium sulfide, ketoconazole. Preferred antipruritics comprise menthol, thymol, oat bran extract, beta glucans, dihydroavenatine, Rosmarinus Officinalis (rosemary) extract, pramoxine, benzocaine, and lidocaine. Preferred hair growth stimulant pharmaceutical ingredients comprise minoxidil, finasteride, spironolactone, platelet-rich plasma, duasteride, and 5-alpha reductase inhibitors. It is preferred not to use linoleic acid in topical hair volumizing compositions and/or treatment methods herein, but it may be used in some embodiments. In other embodiments, one or more of these ingredients may be excluded from treatment compositions and/or methods of treating, such as separate oral doses or topical applications

In other embodiments, the topical volumizing composition may also contain 5-α reductase inhibitors; retinoids; bis-aminopropyl diglycol dimaleate; caffeine and theophylline; Curcuma longa extract; Withania somnifera extract; Serenoa serrulata fruit extract; resveratrol; ubiquinone; Camellia sinensis leaf extract; L-methionine; L-cysteine hydrochloride; pantothenic acid and its derivatives; biotin; selenium; zinc; cholecalciferol; sodium hyaluronate, one or more Chinese botanical extracts of reishi mushrooms, Morus albus, wu wei zin, or fo-ti extracts.

Disodium copper (II) isochlorin e4 or trisodium copper (II) chlorin e6 are labile; over time, they are susceptible to hydrolysis and oxidative, temperature-induced and other types of degradation indicated by change in color value (e.g., CIELAB color space). Stabilization of these compounds is demonstrated by reduction in change from initial color value. Accordingly, in some embodiments, topical hair volumizing compositions of the disclosure comprise one or both of an anionic polysaccharide and/or an antioxidant. Anionic polysaccharides that may be used include cellulosic ethers; carrageenan; kelp; xanthan gum; tragacanth; gum acacia; and/or locust bean gum, but other anionic polysaccharides may also be used in some embodiments. Antioxidants that may be used include tocopherols, tocotrienols, sulfite and bisulfite salts, BHA, BHT, retinoids, superoxide dismutase, cysteine, N-acetyl cysteine, ubidecarenone, glutathione, ascorbic acid or sodium, calcium or potassium ascorbate, tetrahexadecyl ascorbate, ascobyl phosphate, ascorbyl palmitate, hydroxyacetophenone, Trolox®, resveratrol, cysteamine HCL, tert-butylhydroxyquinone, Tris(2,4-di-tert-butylphenyl)phosphite, hydroxytyrosol, epigallocatechin, ergothionine, alpha-arbutin, and octadecyl-di-t-butyl-4-hydroxycinnamate. These anionic polysaccharide and/or antioxidant ingredients are preferably included in amount(s) that aid in stabilizing the disodium copper (II) isochlorin e4 and/or trisodium copper (II) chlorin e6 as measured by a visual comparison and/or a reduction in colorimetric change. Usually, visible comparisons are used for cosmetics where RT (room temperature; 25 degrees+/−2 degrees centigrade) and accelerated temperature (40+/−2 degrees centigrade) conditions are compared to refrigerated samples (4 degrees+/−2 degrees centigrade) for visually perceptible changes; however, colorimetry can also be used. The color stability of compositions comprising copper chlorins may be assessed by measuring the Delta E change (total color value change) at room temperature (25° C.) and at elevated temperature (usually 40° C.) and at a control temperature (usually 4° C.) for a period of two or three months. The 25° and 40° samples are compared to the control at each test period to determine total color change (Delta E). A Delta E of 5 or less for the change at two months at 40° C. versus the control is generally considered acceptable. Various composition formulas may be compared for Delta E change and those with lowest Delta E changes may be preferred for treatment compositions according to some embodiments herein. For drug items, high-performance liquid chromatography coupled to mass spectrometry detection (HPLC/MS) changes or other stability-indicating methods for assay of the active ingredients may be used. Any of these may be used for assessing the stability of treatment compositions according to embodiments herein. For embodiments of a treatment composition that do not include disodium copper (II) isochlorin e4 or trisodium copper (II) chlorin e6, it is not required to use an anionic polysaccharide and/or antioxidant, but they may still be used. In some embodiments of treatment compositions that include disodium copper (II) isochlorin e4 or trisodium copper (II) chlorin e6, antioxidants may be used without an anionic polysaccharide.

Treatment compositions according to some preferred embodiments of the disclosure may comprise the ingredients listed below in Tables 1A-1C.

TABLE 1A
Treatment Composition Ingredients

| | | More |
|---|---|---|
| | Preferred | Preferred |
| Ingredient | % wt/wt | % wt/wt |
| Chelated Copper Chlorin Compound | 0.02-0.1 | 0.03-0.05 |
| Non-Aqueous Solvent | 10-40 | 20-35 |
| Glycol Solvent/Penetrant | 5-30 | 10-15 |
| Surfactant/Emulsifier/Penetrant | 0.1-5 | 0.2-1 |
| Viscosity Control Agent | 0-2 | 0.1-0.5 |
| Antioxidant | 0.01-5.0 | 0.05-0.5 |
| Preservative | 0.1-1.0 | 0.4-0.75 |
| pH adjusting agent* | 0.001-0.5 | 0.05-0.25 |
| Water | QS to 100% | |

*In a quantity as needed to adjust pH between 7.5-8.5

Preferred non-aqueous solvents comprise SD Alcohol, 190 proof (denatured alcohol; ethanol SDA 40B), but others may also be used. Preferred glycol solvents/penetrants include diethylene glycol monoethyl ether, butylene glycol, pentylene glycol, and hexylene glycol, but others may also be used. Preferred surfactant/emulsifier/penetrants include phosphatidylcholine, but others may also be used. Preferred viscosity control agents include Carbomer, carboxymethylcellulose, hydroxypropylcellulose and hydroxyethylcellulose, but others may also be used. Preferred antioxidants include tocopheryl acetate and sodium ascorbate, but others may also be used. Preferred preservatives include phenoxyethanol and phenylethyl alcohol, but others may also be used. Preferred pH adjusting agents include citric acid and sodium hydroxide, but others may also be used. Treatment compositions according to some embodiments have a pH of 7.5 to 8.5, more preferably 8.0 to 8.5.

TABLE 1B
Treatment Composition Ingredients (JPM-01-158)

| Ingredient | % w/w |
|---|---|
| Trisodium Copper (II) Chlorin e6 | 0.075 |
| dl-alpha tocopherol acetate | 0.05 |
| Diethylene Glycol Monoethyl Ether | 5.0 |
| (Transcutol P) | |
| Butylene Glycol | 15.0 |
| SD Alcohol, 95% | 39.0 |
| S75 Lecithin (Lipoid) | 1.0 |
| Citric Acid* | 0.05 |
| Purified Water | 39.825 |

*In a quantity as needed to adjust pH between 7.5-8.0

TABLE 1C
Treatment Composition Ingredients

| Ingredient | % w/w |
|---|---|
| Trisodium Copper (II) Chlorin e6 | 0.075 |
| Diethylene Glycol Monoethyl Ether | 2.4 |
| (Transcutol P) | |
| Butylene Glycol | 15.0 |
| Pentylene Glycol | 7.5 |
| Hexylene Glycol | 7.0 |
| Phospholipon 90G (Phosphatidylcholine; | 0.2 |
| Lipoid) | |
| Sodium Hydroxide Solution, 25%* | 0.1 |
| Ascorbic Acid | 0.1 |
| SDA 40-B 190 Proof Alcohol | 30.0 |
| Sodium Hydroxide Solution, 10%* | qs |
| Citric Acid* | qs |
| Purified Water | 50.125 |

*In a quantity as need to adjust pH between 8.3-8.5

Treatment compositions according to embodiments herein may achieve treatment and/or prevention of age-related HHT or FPHL or MPHL by first upregulating or downregulating certain genes that may otherwise cause premature termination of the anagen phase of the hair growth cycle.

“Biomarkers” are molecular entities which can be detected in a biological sample (from a patient) that are useful in diagnosing and evaluating response to a treatment. Biomarkers include, but are not limited to, DNA, RNA, mRNA, protein.

For purposes of the present invention disclosure, a change of 2-fold (200%) or more in the “expression level” of a biomarker (“expressed genes”) is “significant”.

“Expression level” refers to the amount of a polynucleotide or an amino acid product or protein in a biological sample. “Expressed genes” include those that are transcribed into an RNA and then translated into a protein, as well as those that are transcribed into messenger RNA (mRNA) but not translated into a protein (e.g., transfer and ribosomal RNAs).

In the context of the present disclosure, expression of biomarkers can be detected on levels of mRNA or protein. mRNA can be assessed using well-established methodologies known to the skilled artisan, including PCR (including qRT-PCR, RT-PCR), and cDNA micro-array chip (e.g., “GeneChip” from Affymetrix or the equivalent). Protein levels can be detected using quantification techniques known to the skilled artisan including enzyme-linked immunosorbent assay (ELISA); electrochemical illuminescence via mesoscale discovery (MSD); Western-blot; mass spectrometry; Biacore© which can measure antibody-antigen interactions, protein-protein interactions, protein-DNA interactions.

Probes/detecting agents for detecting biomarkers are known in the art and include oligonucleotide probes/primers, micro-array chip probes, and antibodies specific for the markers. Gene expression microarrays are increasingly used in clinical research to establish biomarker signatures.

A transcriptome study of sodium copper chlorophyllin complex, trisodium copper (II) chlorin e6, trisodium copper (II) chlorin p6, and disodium copper (II) isochlorin e4 was conducted by Sunny Biodiscovery laboratory and Thermo-Fisher Scientific Microarray Research Services Laboratory, both of Santa Clara, California to determine gene regulatory activity of these compounds.

The method used to generate a full transcriptome profile and analysis of genes significantly regulated by each of copper chlorophyllin complex, disodium copper (II) isochlorin e4, trisodium copper (II) chlorin e6, and trisodium copper (II) chlorin e6 is detailed as follows:

Human neonatal epidermal keratinocytes (HEKn) were obtained from Cell Applications (San Diego, CA) and were plated in a 96 well plate (Bioland Scientific, Paramount, CA, item #701003 made of high clarity polystyrene) with keratinocyte growth media (Human EpiVita Serum-Free Growth Medium for Neonatal Cells; Cell Applications, San Diego, CA). Cells were mixed with growth media at a rate of 10,000 cells per 200 μl (approximately 104 cells per well) and added to the 96 wells (0.2 mL/well) and incubated overnight at 37° C.

Following the overnight incubation of keratinocytes, each of the 4 test compounds was separately dissolved in sterile distilled water at a concentration of 20 mg/mL. Dissolution occurs at 20-250C with slight agitation for 1-2 minutes until no solid particles were visible. This stock solution of 20 mg/mL was then further diluted with sterile water to a concentration of 500 ug/mL The 500 ug/mL solution of each test compound was added to the cells in the growth media at a ratio of 1:20 (test solution: cells & media) and incubated in the wells for 24 hours at 370 C. The four test compounds evaluated are listed in Table 2 below:

TABLE 2

| Test Material | Lot number | Abbreviation |
|---|---|---|
| Biocolor Green C4 | SR-013738 | Biocolor C4 |
| NB5637 (Sodium Copper | | |
| Chlorophyllin Complex; | | |
| FMC Corp, Centrum 100, | | |
| Burton upon Trent DE14 | | |
| 2WD UK) | | |
| Disodium Copper (II) | DC19-12923 | Chlorin e4 |
| Isochlorin e4 | | |
| (Frontier Scientific, | | |
| Logan, UT) | | |
| Trisodium Copper (II) | RP14-8608 | Chlorin e6 |
| Chlorin e6 | | |
| (Frontier Scientific, | | |
| Logan, UT) | | |
| Trisodium Copper (II) | DC19-12924 | Chlorin p6 |
| Chlorin p6 | | |
| (Frontier Scientific, | | |
| Logan, UT) | | |

The following day the test solution and growth media were removed from the wells and the cells were rinsed with phosphate-buffered saline (PBS).

The RNA from the cells treated with each of the test compounds was extracted and purified with a RNeasy Mini Plus Kit (Catalogue #74134; Qiagen, Germantown, MD) with automated lysing and homogenization using a Qiacube Connect robotic station (Qiagen, Germantown, MD).

The protocol for RNA extraction is selected on the Qiacube Connect automated station and cell samples, labware, and reagents are loaded as instructed. Cells are first lysed and homogenized in highly denaturing guanidine-isothiocyanate-containing Buffer RLT Plus, which immediately inactivates RNases to ensure isolation of intact RNA. The lysate is passed through Qiacube Connect loaded gDNA Eliminator spin columns, ethanol is added to the flow-through, and the samples are then applied to Qiacube preloaded RNeasy MmFElute spin columns. RNA binds to the membrane and contaminants are washed away. High quality RNA is eluted and collected in a total of 30 uL sterile distilled water.

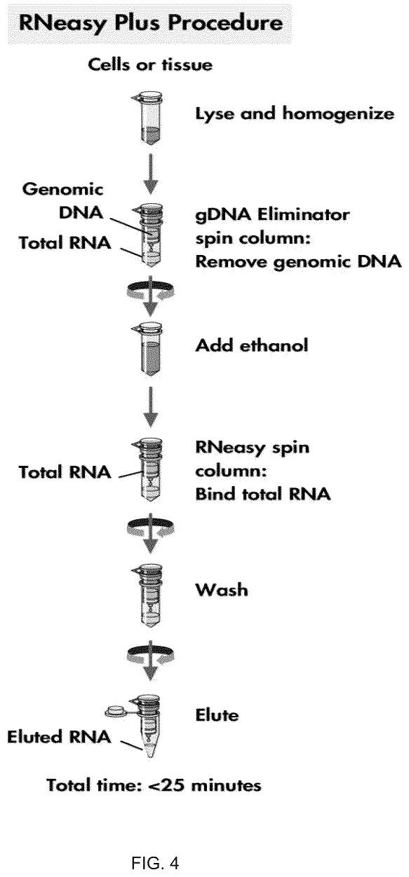

The stepwise procedure for production of RNA using RNeasy Plus procedure is outlined in the schematic in FIG. 4.

Once collected, the purity of the RNA is assessed with the NanoDrop Lite spectrophotometer (Thermo Fisher Scientific, Waltham, MA) at 260 nm and 280 nm.

The NanoDrop Lite unit is a small, stand-alone UV spectrophotometer that is designed to measure microvolumes (1-2 uL) of nucleic acid and protein samples. Purity of the collected RNA was verified by adding 1 uL of the collected RNA to the NanoDrop Lite unit, selecting the RNA application on the NanoDrop Lite Home screen and following the stepwise measuring procedure as detailed in the NanoDrop Lite Manual. The sample purity is displayed by the NanoDrop Lite screen based on the A260/A280 ratio.

The extracted RNA for each of the 4 test compounds from the above procedure was then transported on dry ice to a laboratory specializing in transcriptome analysis (Thermo Fisher Scientific Microarray Research Services Laboratory, Santa Clara, CA). The transcriptome profiling of the RNA samples was conducted using the Clariom S GeneChip® Pico Assay platform. The resulting CHP files containing probe set analysis results generated with Affymetrix® software were uploaded and differential gene expression, as well as functional interaction networks were analyzed with Summarization SST-RMA algorithm using the Affymetrix TAC® (Transcriptome Analysis Console) software version 4.0.2.15 (Applied Biosystems by Thermo Fisher).

The Human Clariom S Assay design provides extensive coverage of all known well-annotated genes and accurately measures gene-level expression from >20,000 well-annotated genes and can utilize RNA from various sample types including blood, cells, and fresh/fresh-frozen or FFPE tissues. Human Clariom S Assays detect only the exons present in all known transcript isoforms expressed from a single gene locus-constitutive exon. This differs from other gene-level array technologies and shallow RNA-Seq, which provide either a biased view of gene expression or data that are complicated by variation in expression of transcript variants. By detecting only constitutive exons throughout the length of each known gene, Human Clariom S Assays generate the most accurate and truest measurement of gene-level expression. Clariom S solutions are formats for single-sample (cartridge array) processing on the GeneChip™ 3000 instrument system or the fully automated GeneTitan® instrument (Thermo Fisher).

Excel® spreadsheets and CHP and CEL files of genes affected by each compound in Table 2 were generated by Thermo Fisher Microarray Research Services Laboratory based on the above process. The CHP or CEL files generated by Thermo Fisher were then imported into the TAC® software console to identify genes significantly downregulated or genes significantly upregulated by the compounds in Table 2 above. In reporting the gene regulation, results may show a plus sign (+) or no sign (i.e., a lack of a plus sign and a lack of a minus sign) to indicate upregulation (particularly when the words “upregulated” or “upregulation” are not specifically used) and may show a minus sign (−) to indicate downregulation (particularly when the words “downregulated” or “downregulation” are not specifically used), as shown in the listing of results in Table 4. Significant upregulation is generally upregulation of 200% or more, which may be expressed as +200% or 200% or more (e.g., 210%, 250%, 300%, etc.) or as folding of +2 or 2 or more (e.g., 2.1, 2.5, 3.0, etc.). Significant downregulation is generally downregulation of 200% or more, which may be expressed as −200% or more (e.g., −210%, −250%, −300%, etc.) or as folding of −2 or more (e.g., −2.1, −2.5, −3.0, etc.). For example, “regulated by −200%” and “downregulated by 200%” have the same meaning. Likewise, “regulated by 200%,” “regulated by +200%,” and “upregulated by 200%” have the same meaning.

Treatment compositions according to embodiments herein were found to regulate the expression of certain genes based on the described transcriptomic testing wherein 20 ug/mL of trisodium copper chlorin e6 or disodium copper isochlorin e4 or trisodium copper chlorin p6 or sodium copper chlorphyllin complex or sterile distilled water was added to normal keratinocyte cell cultures (HEKn cells) for 24 hours and gene expression of each copper chlorin compound and the copper chlorophyllin complex was compared to the water control. (Sunny Biodiscovery Transcriptome Study 1085)

Differential analysis of the transcriptome data indicated that treatment compositions according to embodiments herein may, and preferably do, significantly increase or decrease expression of one or more genes that code for at least one, preferably at least two, more preferably at least three, even more preferably at least four, and most preferably at least five of the following proteins: ARNTL2; BAMBI; CCL20; CSF1; CSF2; CSF3; CTSB; CXCL1; CXCL11; CXCL2; CXCL3; CXCL8; DDX58; DDX60; DDX60L; DKK1; EGR1; EIF2AK2; EPST11; EREG; FGF2; FN1; FOS; GAB1; GADD45B; GDF15; GPX2; HAS3; HBEGF; HERC5; HERC6; HIF1AN; HSP90B1; CMPK2; ICAM1; IFI27; IFI44; IFI44L; IFI6; IFIH1; IFIT1; IFIT2; IFIT5; IGFBP3, IL1A; IGHA1; IL1B; IL6; JUN; JUNB; LY6E; MAFF; MMP8; MYC; NAB1; FGF2; OAS2; OAS3; OASL; OVOL1; PARP 9; PARP12; PARP14; PCDH1; PLAUR; PLSCR1; RSAD2; PTGFRN; PTGS2; RHOB; RHOB; RTP4; SAMD9L; SERPINB2; TGFB1; SERPING1; SIGLEC1; SKP2; SPATS2L; STAT1; TGFB2; TGFB2-OT1; TNF; TNFAIP6; TNFSF12; SOD2; USP18; USP41; VEGFA; XAF1. Such increase or decrease in expression of genes that code for these proteins is measured at around 24 hours after application of a treatment composition according to an embodiment herein and the increased or decreased gene expression was considered significant if the fold change in the gene is preferably 200% or more

The effect of trisodium copper (II) chlorin e6 (TCCE6) and a prodrug of copper chlorin e6; diethyl ester of copper (II) chlorin e6 (DECCE6), were further evaluated for gene expression of normal keratinocytes (HEKn cells) using specific gene primers from the previous list of genes identified by the 1085 transcriptome study.

The TCCE6 and DECCE6 test materials were stored at −20° C. until used. The day of the experiment, stock solution of each test material was prepared in DMSO at 20 mg/ml followed by further dilution to 400 μg/ml in sterile distilled H₂O. The diluted test material as well as the negative control (H₂O) were distributed 50 μl into each well containing cells in 950 μl culture medium. The final dose of the test material contacting cells was 20 μg/ml.

Normal human keratinocytes (Table 3) were plated in a 24-well plate at 60,000 cells/well in Keratinocyte Growth Medium (KM-2; ZenBio, Durham, North Carolina Lot #12225). Next day test materials were added and the incubation was pursued for 24 hours at 37° C. and 5% CO₂.

TABLE 3
Cells used.

| Cells | Supplier | Cat# | Lot# | Passage |
|---|---|---|---|---|
| HEKa 28 yr F | Cell Applications | 102-05a | 3260 | 4 |

After 24 hours of the cell culture incubation, RNA was extracted with RNeasy Mini kit cat #74104 from Qiagen (Germantown, MD), using QiaCube Connect robotic station (Qiagen). Purified total RNA was assessed at 260 & 280 nm with NanoDrop Lite (Thermo Fisher Scientific, Waltham, MA). For qPCR reactions, cDNA was prepared using High-Capacity RNA-to-cDNA Kit (cat. #4387406, ThermoFisher) and the expression of the genes of interest (Table 4) was measured by real-time quantitative PCR with C1000 Touch System with CFX384 optical module, using PCR primers from RealTimePrimers (Elkins Park, PA) and Forget-Me-Not EvaGreen qPCR Master Mix (Low ROX) from Biotium (Fremont, CA; Lot #23F1213). Efficiency ΔΔCₜ method was used for quantification of results, after the normalization of gene expression to the housekeeping genes GAPDH and HPRT1. Ct (cycle threshold) is defined as the number of replication cycles required for the fluorescent signal to exceed the background level. Ct levels are inversely proportional to the amount of target nucleic acid in the sample (i.e., the lower the Ct level the greater the amount of target nucleic acid in the sample).

In this particular study, genes were considered differentially expressed if the p value determined by the two-tailed t-test was <0.05, and the modulation or fold change was >1.7. Gene ontology enrichment/depletion analysis was also performed wherever applicable using the Metascape online platform (Zhou et al. Nature Commun. 2019; 10:1523).

PCR primers were used to interrogate the expression of the following panel of genes: AREL1; ARRDC3; AQP3; BAMBI; CCL20; CDKN1A; CCL20; CXCL1; CXCL2; CXCL3; CXCL8; CXCL10; CYB561; CYP1A1; CSF1; CSF2; DDX5; DEFB1; EPGN; FGF2; FOS; G2E3; GDF15; GPX2; HBEGF; HERC1; HECTD1; HIF1AN; HSP90B1; IFI44L; JUN; JUNB; LTB4R: LTB4R2; MAFF; MAP1LC3B; MAPK14; MGAT1; MGAT5; MOGS; N4BP1; NFKBIA; PELI1; PLAUR; PPP1R15A; PTGFRN; PTGS2; PTX3; REL; RSAD2; SAMD9L; SPRR2B; SOD2; TGFB1; TNF; TNFAIP3; UBE2S; UBE20; UBE3B; UBE4B; VEGFA; VCP, GAPDH (HKG), HPRT1 (HKG), 18S (HKG). The HKG designation refers to housekeeping genes.

Sufficient quantities of high purity RNA were extracted from all samples and all experimental conditions were found to elicit a strong gene expression—modulatory response, with the statistically significant effect on >50% of the genes in the tested PCR array. Most gene responses were geared towards immunostimulatory effects. Table 4 also lists the expression Fold Change for all tested genes vs. water-treated control and FIGS. 5 and 6 shows Gene Ontology (GO) terms most enriched in cell populations treated with trisodium copper (11) chlorin e6 or with the diethyl ester of copper (11) chlorin e6.

TABLE 4
Differential Gene Expression of trisodium copper (II0 chlorin
e6 (TCCE6) and Diethyl ester of copper (II) chlorin e6 (DECCE6)

| | DECCE6 R36097 | | | | TCCE6 C40405 | | |
|---|---|---|---|---|---|---|---|
| | 20 ug/mL | | | | 20 ug/mL | | |
| | | Fold Up- | | | | Fold Up- | |
| Gene | T-TEST | or Down- | | Gene | T-TEST | or Down- | |
| Symbol | p value | Regulation | Comments* | Symbol | p value | Regulation | Comments* |
| 18S | 0.002 | 1.2 | OKAY | 18S | 0.002 | 1.1 | OKAY |
| AQP3 | 0.035 | 5.5 | OKAY | AQP3 | 0.002 | 1.7 | OKAY |
| AREL1 | 0.001 | −5.8 | A | AREL1 | 0.742 | −1.0 | OKAY |
| ARRDC3 | 0.037 | 1.9 | A | ARRDC3 | 0.001 | 6.1 | OKAY |
| BAMBI | 0.335 | 1.3 | B | BAMBI | 0.001 | 6.2 | A |
| CCL20 | 0.000 | 2.6 | OKAY | CCL20 | 0.001 | 1.3 | OKAY |
| CDKN1A | 0.029 | −1.4 | OKAY | CDKN1A | 0.010 | 1.5 | OKAY |
| CSF1 | 0.562 | 1.1 | B | CSF1 | 0.234 | −1.3 | B |
| CSF2 | 0.005 | −1.1 | OKAY | CSF2 | 0.197 | −1.2 | B |
| CXCL1 | 0.001 | 2.7 | OKAY | CXCL1 | 0.030 | 1.6 | OKAY |
| CXCL10 | 0.033 | 7.6 | C | CXCL10 | 0.345 | 2.0 | B |
| CXCL2 | 0.001 | 3.0 | OKAY | CXCL2 | 0.192 | 1.2 | OKAY |
| CXCL3 | 0.762 | −1.1 | A | CXCL3 | 0.197 | 1.7 | OKAY |
| CXCL8 | 0.168 | 1.3 | OKAY | CXCL8 | 0.603 | 1.1 | OKAY |
| CYB561 | 0.661 | −1.2 | A | CYB561 | 0.456 | −1.1 | OKAY |
| CYP1A1 | 0.003 | 2.8 | A | CYP1A1 | 0.006 | −1.8 | A |
| DDX5 | 0.000 | 2.1 | OKAY | DDX5 | 0.298 | 1.0 | OKAY |
| DEFB1 | 0.000 | 3.4 | OKAY | DEFB1 | 0.001 | 2.2 | OKAY |
| EPGN | 0.228 | 1.2 | OKAY | EPGN | 0.000 | 2.3 | OKAY |
| FGF2 | 0.007 | −2.1 | A | FGF2 | 0.101 | −1.2 | OKAY |
| FOS | 0.050 | 15.9 | A | FOS | 0.003 | 3.7 | A |
| G2E3 | 0.028 | 4.1 | A | G2E3 | 0.043 | −1.2 | OKAY |
| GDF15 | 0.064 | 1.7 | OKAY | GDF15 | 0.045 | 1.9 | OKAY |
| GPADH | 0.006 | −1.9 | OKAY | GPADH | 0.150 | −1.2 | OKAY |
| GPX2 | 0.197 | 5.5 | B | GPX2 | 0.201 | −1.1 | B |
| HBEGF | 0.000 | 3.1 | OKAY | HBEGF | 0.001 | 2.5 | OKAY |
| HECTD1 | 0.098 | 1.2 | OKAY | HECTD1 | 0.800 | −1.0 | OKAY |
| HERC1 | 0.009 | 3.5 | A | HERC1 | 0.398 | 1.1 | OKAY |
| HIF1AN | 0.060 | −1.6 | A | HIF1AN | 0.072 | −1.3 | OKAY |
| HPRT1 | 0.017 | 1.6 | A | HPRT1 | 0.744 | 1.0 | OKAY |
| HSP90B1 | 0.019 | 1.2 | OKAY | HSP90B1 | 0.035 | 1.4 | OKAY |
| IFI44L | 0.006 | 3.9 | OKAY | IFI44L | 0.026 | −2.5 | OKAY |
| JUN | 0.000 | 3.3 | OKAY | JUN | 0.176 | 1.2 | OKAY |
| JUNB | 0.000 | 1.6 | OKAY | JUNB | 0.000 | 1.9 | OKAY |
| LTB4R | 0.172 | −1.3 | A | LTB4R | 0.569 | 1.1 | OKAY |
| LTB4R2 | 0.131 | −1.7 | B | LTB4R2 | 0.014 | 1.9 | OKAY |
| MAFF | 0.309 | −1.1 | A | MAFF | 0.001 | 2.2 | OKAY |
| MAP1LC3B | 0.437 | 1.2 | OKAY | MAP1LC3B | 0.001 | 1.5 | OKAY |
| MAPK14 | 0.000 | −1.7 | A | MAPK14 | 0.021 | −1.1 | OKAY |
| MGAT1 | 0.756 | −1.0 | B | MGAT1 | 0.016 | −1.7 | OKAY |
| MGAT5 | 0.000 | −1.8 | A | MGAT5 | 0.014 | 1.2 | OKAY |
| MOGS | 0.002 | −2.8 | A | MOGS | 0.000 | −1.3 | OKAY |
| N4BP1 | 0.002 | 1.7 | OKAY | N4BP1 | 0.009 | 1.3 | OKAY |
| NFKBIA | 0.000 | 4.6 | OKAY | NFKBIA | 0.019 | 1.3 | OKAY |
| PELI1 | 0.042 | −1.5 | A | PELI1 | 0.109 | 1.4 | OKAY |
| PLAUR | 0.002 | 2.1 | OKAY | PLAUR | 0.004 | 9.7 | OKAY |
| PPP1R15A | 0.000 | 3.2 | OKAY | PPP1R15A | 0.005 | 2.5 | OKAY |
| PTGFRN | 0.000 | −4.6 | A | PTGFRN | 0.001 | −2.6 | OKAY |
| PTGS2 | 0.447 | −1.1 | OKAY | PTGS2 | 0.000 | 2.4 | OKAY |
| PTX3 | 0.139 | 1.1 | B | PTX3 | 0.192 | 1.8 | B |
| REL | 0.026 | 1.6 | A | REL | 0.380 | −1.1 | OKAY |
| RSAD2 | 0.007 | 1.9 | OKAY | RSAD2 | 0.183 | 1.2 | OKAY |
| SAMD9L | 0.233 | 1.3 | B | SAMD9L | 0.141 | 1.5 | B |
| SOD2 | 0.076 | 1.2 | OKAY | SOD2 | 0.017 | −1.4 | OKAY |
| SPRR2B | 0.028 | 2.4 | OKAY | SPRR2B | 0.007 | 6.5 | OKAY |
| TGFB1 | 0.000 | −1.9 | A | TGFB1 | 0.104 | −1.2 | OKAY |
| TNF | 0.009 | 16.4 | OKAY | TNF | 0.408 | 1.2 | B |
| TNFAIP3 | 0.009 | 2.4 | OKAY | TNFAIP3 | 0.008 | 2.6 | OKAY |
| UBE20 | 0.005 | 1.8 | OKAY | UBE20 | 0.005 | 2.5 | OKAY |
| UBE2S | 0.034 | 2.2 | OKAY | UBE2S | 0.632 | −1.1 | B |
| UBE3B | 0.069 | 1.2 | A | UBE3B | 0.005 | 1.4 | OKAY |
| UBE4B | 0.060 | 1.2 | A | UBE4B | 0.156 | −1.4 | A |
| VCP | 0.005 | −2.0 | OKAY | VCP | 0.113 | 1.2 | OKAY |
| VEGFA | 0.033 | 1.5 | OKAY | VEGFA | 0.006 | 2.5 | OKAY |

*Comments: OKAY: high (<30 cycles to detection) gene expression; A: low (≥30 cycles to detection) gene expression in one of the comparators, B: low gene expression in both comparators; C: gene expression not detected; .18S, HPRT1, & GAPDH are housekeeping genes

Based on Table 4 results associated with the use of gene primers listed, trisodium copper chlorin e6 was found to significantly (p<0.05), upregulate or downregulate the following genes based on fold value changes of 2.0× (200%) or greater: ARRDC3, BAMBI, CXCL10, DEFB1; EPGN; FOS; HBEGF; IFI44L; MAFF; PLAUR; PPP1R15A; PTGS2; SPRR2B; TNFAIP3; UBE20; and VEGFA.

Treatment compositions according to embodiments herein may, and preferably do, significantly increase or decrease expression of one or more genes that code for at least one, preferably at least two, more preferably at least three, even more preferably at least four, and most preferably at least five of the following proteins: ARRDC3, BAMBI, CXCL10, DEFB1; EPGN; FOS; HBEGF; IFI44L; MAFF; PLAUR; PPP1R15A; PTGS2; SPRR2B; TNFAIP3; UBE20; and VEGFA.

The application of a treatment composition of an embodiment of the disclosure for one or more days may result in gene regulation changes noted above based on in vitro transcriptome and gene primer analysis of keratinocytes exposed to 20 ug/mL of copper chlorin compounds for 24 hours.

The effect of the daily application of embodiments herein for a period of up to six months or less may result in a change in the immune characteristics of the skin and hair follicle areas treated as measured by differential transcriptomic analysis of skin and follicular tissue before and after treatment with treatment compositions herein. Next-generation sequencing (NGS) and differential gene analysis was conducted by Azenta Life Sciences (Burlington, MA) on scalp biopsy samples from FPHL subjects treated daily for 6 months with compositions of the embodiment. The biological change in the area of thinning terminal hairs treated with treatment compositions herein is characterized by primarily a reduction in expression of immunoglobulin and certain chemokine genes. A reduction in the immunoglobulin antibodies of the skin and hair follicles treated by embodiments herein daily for a period of 6 months or less may be an indication of the improvement or restoration of the immune privilege of the hair follicles of areas of age-related HHT, AGA or FPHL and MPHL.

Immunoglobulins, or multi-subunit proteins, consist of two identical heavy chains (IgH) and two identical light chains (IgL). They are divided by the amino acid sequence of their heavy chains into five classes (Immunoglobulin A; Immunoglobulin D; Immunoglobulin E; Immunoglobulin G; Immunoglobulin M) and various subclasses. IgH chains contain a series of domains, usually a variable domain (IGHV) that is important for binding antigen, and several constant domains, including immunoglobulin heavy constant delta (IGHD).

Treatment compositions according to embodiments herein, when used for a period of 6 months or less may decrease follicle microinflammation antigens and thereby result in a secondary immune response of the human hair follicles and surrounding tissue to downregulate the expression of at least one gene that codes for human immunoglobulin kappa variable (IGVK) protein(s), human immunoglobulin lambda variable (IGVL) protein(s), human immuno-globulin heavy chain (IgH) protein(s), or a combination thereof. In some embodiments, treatment compositions downregulate these genes by at least 200%. The differential gene expression generated in testing of treatment compositions herein was a comparison at baseline (day 1 of the clinical, prior to the first application of a treatment composition) and after a treatment period of 24 weeks with a daily application of a treatment composition herein.

In some embodiments, treatment compositions, when topically applied for 6 months, herein result in the downregulation of one or more of (i) IGKV1 D-16; (ii) IGKV3-20; (iii) IGKV3-11; (iv) IGKV2D-28; or (v) IGKV2D-28. In other embodiments, treatment compositions and/or methods herein result in the downregulation of each of: (i) IGKV1 D-16; (ii) IGKV3-20; (iii) IGKV3-11; (iv) IGKV2D-28; and (v) IGKV2D-28.

In some embodiments, treatment compositions herein result in the downregulation of one or more of: (i) IGLV3-19; (ii) IGLV1-40; (iii) IGLV1-51; (iv) IGLV2-11; or (v) IGLV1-51. In other embodiments, treatment compositions and/or methods herein result in the downregulation of each of: i) IGLV3-19; (ii) IGLV1-40; (iii) IGLV1-51; (iv) IGLV2-11; and (v) IGLV1-51.

In some embodiments, treatment compositions herein result in the downregulation of: (a) one or more of (i) IGKV1 D-16; (ii) IGKV3-20; (iii) IGKV3-11; (iv) IGKV2D-28; or (v) IGKV2D-28; and (b) one or more of (i) IGLV3-19; (ii) IGLV1-40; (iii) IGLV1-51; (iv) IGLV2-11; or (v) IGLV1-51. In some embodiments, treatment compositions herein result in the downregulation of at least one gene in category (a) by at least 200% and downregulate at least one gene in category (b) by at least 200%. In other embodiments, treatment compositions herein result in the downregulation of at least two genes in category (a) by at least 200% and the downregulation of at least two genes in category (b) by at least 200%. In still other embodiments, treatment compositions and/or methods herein result in the downregulation of at least three genes in category (a) by at least 200% and the downregulation of at least three genes in category (b) by at least 200%.

In still other embodiments, treatment compositions herein result in the downregulation of one or more of: (i) IGHV3-48; (ii) IGHV3-30; (iii) IGHV3-53; or (iv) IGHV3-7. In other embodiments, treatment compositions herein result in the downregulation of each of: (i) IGHV3-48; (ii) IGHV3-30; (iii) IGHV3-53; and (iv) IGHV3-7.

In some embodiments, treatment compositions herein result in the downregulation of: (a) one or more of (i) IGKV1 D-16; (ii) IGKV3-20; (iii) IGKV3-11; (iv) IGKV2D-28 or (v) IGKV2D-28; and (b) one or more of (i) IGHV3-48; (ii) IGHV3-30; (iii) IGHV3-53; or (iv) IGHV3-7. In some embodiments, treatment compositions herein result in the downregulation of at least one gene in category (a) by at least 200% and downregulation at least one gene in category (b) by at least 200%. In other embodiments, treatment compositions herein result in the downregulation of at least two genes in category (a) by at least 200% and downregulation of at least two genes in category (b) by at least 200%. In still other embodiments, treatment compositions herein result in the downregulation of at least three genes in category (a) by at least 200% and downregulation of at least three genes in category (b) by at least 200%.

In another embodiment, treatment compositions herein result in the downregulation of (a) one or more of: (i) IGLV3-19; (ii) IGLV1-40; (iii) IGLV1-51; (iv) IGLV2-11; or (v) IGLV1-51; and (b) one or more of (i) IGHV3-48; (ii) IGHV3-30; (iii) IGHV3-53; and (iv) IGHV3-7. In some embodiments, treatment compositions herein result in the downregulation of at least one gene in category (a) by at least 200% and downregulation of at least one gene in category (b) by at least 200%. In other embodiments, treatment compositions herein result in the downregulation of at least two genes in category (a) by at least 200% and downregulation of at least two genes in category (b) by at least 200%. In still other embodiments, treatment compositions herein result in the downregulation of at least three genes in category (a) by at least 200% and downregulation of at least three genes in category (b) by at least 200%.

In another embodiment, treatment compositions herein result in the downregulation of (a) one or more of (i) IGLV3-19; (ii) IGLV1-40; (iii) IGLV1-51; (iv) IGLV2-11; or (v) IGLV1-51; and (b) one or more of (i) IGLV3-19; (ii) IGLV1-40; (iii) IGLV1-51; (iv) IGLV2-11; and (v) IGLV1-51; and (c) one or more of (i) IGHV3-48; (ii) IGHV3-30; (iii) IGHV3-53; and (iv) IGHV3-7. In some embodiments, treatment compositions herein result in the downregulation of at least one gene in category (a) by at least 200% and downregulation of at least one gene in category (b) by at least 200% and downregulation of at least one gene in category (c) by at least 200%. In other embodiments, treatment compositions herein result in the downregulation of at least two genes in category (a) by at least 200% and downregulation of at least two genes in category (b) by at least 200% and downregulation of at least two genes in category (c) by at least 200%. In still other embodiments, treatment compositions herein result in the downregulation of at least three genes in category (a) by at least 200% and downregulation of at least three genes in category (b) by at least 200% and downregulation of at least three genes in category (c) by at least 200%.

Treatment compositions according to embodiments herein, when used for a period of 6 months or less may decrease inflammatory antigens and thereby result in a secondary immune response of the human hair follicles and surrounding tissue as measured by differential transcriptomic analysis. The differential change in gene expression measured is primarily downregulation of certain immunoglobulin genes previously described but may also include downregulation of certain chemokines and upregulation of certain immunoglobulins.

A decrease in inflammatory antigens may result in a secondary immune response of the human hair follicles and surrounding tissue by downregulation of the expression of at least one chemokine gene that codes for: (i) PRSS21; (ii) GZMK; (iii) ICOS; (iv) CXCL13; (v) CXCL10; or (vi) CCL24 when measured 6 months after daily application of a treatment composition according to an embodiment herein comprising a chlorin and preferably a copper chlorin.

Treatment compositions according to embodiments herein, when measured 6 months after daily application, causes a secondary immune response that includes upregulating of the expression of gene(s) that code for one or both of the following IGLV proteins: IGLV2-8 and/or IGLV2-23. In some embodiments, treatment compositions and/or methods herein upregulate these genes by at least 200%.

Differential changes in the expression of genes—downregulation and upregulation—as described herein is assessed by analysis of biopsied treated tissue after the end of a treatment period, preferably a treatment period of at least six months comprising once-daily application to a hair area of hair thinning (such as the scalp) of a treatment composition according to an embodiment herein.

### Treatment Methods

In some embodiments, a method of treating and/or preventing HHT, AGA, or FPHL and MPHL, or of restoring HHF IP, comprises the step of applying a hair volumizing composition comprising a chlorin to a hair area. A “hair area” is used herein to refer to any human skin area of the body and/or the scalp comprising terminal hair or covered with terminal hair or that should comprise or be covered with terminal hair, but has experienced HHT. In some embodiments, a treatment composition comprising a chlorin, and preferably a copper chlorin, is topically applied to a hair area.

In certain preferred embodiments, a topical hair volumizing composition used in methods herein is a treatment composition according to an embodiment disclosed herein.

In practicing the methods of the present invention, a treatment composition (or a hair volumizing composition) comprising a chelated copper chlorin compound may be applied once or several times daily, or at different intervals (e.g., several days per week), and/or at different doses. A hair volumizing composition comprising a chlorin, and preferably a copper chlorin, may, for example, be applied: once daily; once every two days; once every three days; once every four days; once every five days; once every six days; once per week; over the course of a week, for two consecutive days followed by one, two, three, four or five “off” days (references to “off” days mean days without any application of a treatment composition and references to “on” days mean days with one or more applications of a treatment composition); over the course of a week, for three consecutive days followed by one, two, three, or four off days; over the course of a week, for four consecutive days followed by one, two, or three off days; over the course of a week, for five consecutive days followed by one or two off days; over the course of a week, for six consecutive days followed by one off days; two days/week; three days/week; four days/week; five days/week; six days/week. Any combination of on and off days may be used over the course of a treatment period. A treatment period may be anywhere from 1 to 90 days, 1 to 180 days, or longer. Preferred treatment periods are at least 3 months and more preferably at least 6 months. A treatment course may comprise multiple treatment periods, with or without periods of no treatment in between. An application rate may vary over the course of a treatment period or a treatment course and may include off days or weeks (without any treatment) intermixed with on days (with applications one or multiple times per day and/or multiple days per week). Any combination of a number of treatment application doses in any periodic timing within a treatment period may be used with or without days of no treatment application.

To achieve an increase of average density of terminal hairs of greater than 15%, a topical hair volumizing composition comprising a chlorin, and preferably a copper chlorin, in accordance with some embodiments of the disclosure is preferably applied once daily for at least 70 days, preferably for at least 75 days, more preferably for at least 80 days, still more preferably for at least 85 days. According to other embodiments, such application in each case is without skipping application for two or more consecutive days during a treatment period.

To achieve an increase of average diameter of terminal hairs of greater than 5%, a topical hair volumizing composition comprising a chlorin, and preferably a copper chlorin, in accordance with some embodiments of the disclosure is preferably applied once daily for at least 70 days, preferably for at least 75 days, more preferably for at least 80 days, still more preferably for at least 85 days. According to other embodiments, such application in each case is without skipping application for two or more consecutive days during a treatment period.

To achieve an increase of average density of terminal hairs of greater than 25%, a topical hair volumizing composition comprising a chlorin, and preferably a copper chlorin, in accordance with some embodiments of the disclosure is preferably applied once daily for at least 150 days, preferably for at least 155 days, more preferably for at least 160 days, still more preferably for at least 165 days, even more preferably for at least 170 days. According to other embodiments, such application in each case is without skipping application for two or more consecutive days during a treatment period.

To achieve an increase of average density of terminal hairs of greater than 25%, it is especially preferred that a topical hair volumizing composition comprising a chlorin, and preferably a copper chlorin, in accordance with some embodiments of the disclosure be applied once daily for at least 175 days, and even more preferably for at least 180 days. According to other embodiments, such application in each case is without skipping application for two or more consecutive days during a treatment period.

To achieve an increase of average diameter of terminal hairs of greater than 15%, a topical hair volumizing composition comprising a chlorin, preferably a copper chlorin, in accordance with some embodiments of the disclosure is preferably applied once daily for at least 150 days, preferably for at least 155 days, more preferably for at least 160 days, still more preferably for at least 165 days, even more preferably for at least 170 days. According to other embodiments, such application in each case is without skipping application for two or more consecutive days during a treatment period.

To achieve an increase of average diameter of terminal hairs of greater than 15%, it is especially preferred that a topical hair volumizing composition comprising a chlorin, and preferably a copper chlorin, in accordance with some embodiments of the disclosure be applied once daily for at least 175 days, and even more preferably for at least 180 days According to other embodiments, such application in each case is without skipping application for two or more consecutive days during a treatment period.

Dosing and frequency of methods of the present disclosure can and will vary depending on whether, in addition to application of a topical hair volumizing composition according to an embodiment herein, any additional treatment step(s) is/are practiced concomitantly. Additional treatment steps may include one or a combination of (i) administering 6-piperidin-1-ylpyrimidine-2,4-diamine 3-oxide, (ii) administering a 5-α reductase inhibitor selected from the group of 4-azasteroid compounds (such as finasteride and dutasteride), or aldosterone receptor antagonists (such as spironolactone), or a combination thereof, (iii) administering a retinoid (iv) injecting platelet-rich plasma, (v) injecting a corticosteroid, (vi) microneedling or photobiostimulation by low-level laser (light) therapy (LLLT) or light emitting diodes (LED) as described in the following scientific publications, the disclosures of which are incorporated, in pertinent part, herein by reference: Avci, P. et al, in Lasers Surg Med. 2014 February; 46(2): 144-151; Leavitt M. et al, Clin Drug Investig. 2009; 29(5):283-292; Barolet D., Semin Cutan Med Surg. 2008; 27(4):227-238 Clin Drug Investig. 2009; 29(5):283-292.

Retinoids that may be used as an additional treatment include any of a group of compounds including retinoic acid (RA), its active metabolites all-trans-retinol, Tretinoin (all-trans-retinoic acid), Isotretinoin (13-cis-retinoic acid), Alitretinoin (9-cis-retinoic acid), and can be non-aromatic, monoaromatic or contain a cyclic polyene side chain. Non-limiting examples include Retinyl Palmitate, Retinaldehyde, Tazarotene, Adapalene.

Microneedling (also known in the art as “dermarolling”) that may be used as an additional treatment is generally a minimally invasive procedure involving the induction of percutaneous wounds by rolling a dermaroller—a circular array of microneedles (e.g., 0.5-3 mm in length and 0.1-0.25 mm in diameter) studded on drum-shaped cylinder (e.g., 192 microneedles in 8 rows, each row having 24 microneedles) over an area of skin, creating micropunctures in the stratum corneum.

In another embodiment, the treatment method composition of an embodiment herein may contain marine sponge spicules of one or more diactine, triactine, hexactine, tetracline, and polyactine spicule sponge types to enhance the penetration and overall treatment result of the treatment compositions of the embodiment. In other embodiments, one or more of these ingredients may be excluded from treatment compositions and/or methods of treating herein.

In another embodiment, a treatment method comprises applying a treatment composition of an embodiment herein or a solution comprising a chlorin, preferably a copper chlorin, to hair area with a dropper, sachet applicator, sponge, non-woven or natural fiber cloth, glass, metal, plastic or other applicator, metered or non-metered pump spray, metered or non-metered aerosol spray, or manually with a gloved or ungloved human finger. An amount of a treatment composition according to embodiments herein that is applied to hair area is around 0.0005 to 0.002 mL/cm² of hair scalp area of around 700 cm² being treated. An amount of copper chlorin topically applied to each such hair area ranges from about 50-2000 μg, more preferably 100-1500 μg, and most preferably 200-1000 μg.

Methods of applying a treatment composition herein may reduce termination of the anagen phase by downregulating or upregulating the expression of genes that code for certain proteins, as previously discussed with treatment composition embodiments herein. A method of application that may achieve the downregulation or upregulation according to some embodiments comprises topical application of a treatment composition to a hair area of a person having HHT, AGA, FPHL or MPHL at least once daily, for at least 3 months, preferably at least 6 months, with the treatment composition being in a leave-on (or leave-in) form wherein the hair area is not shampooed or wetted for at least 6 hours, more preferably at least 8 hours after application of the treatment composition.

### EXAMPLES

Described in the below examples are treatment compositions and methods according to some embodiments of the disclosure and the results of testing such. The following examples are provided for illustration and are not intended to limit the scope of the disclosure.

### Example 1—Pilot Clinical Study for Improvements in Hair Density/Thickness

Seven otherwise healthy female adults diagnosed with FPHL and HHT based on clinical assessment by an experienced dermatologist experienced in clinical studies of alopecia and diagnosed with FPHL based on either Sinclair (grade II-V) or Ludwig (grade I-Ill) alopecia scales and confirmation with trichoscopy of anisotrichosis; having as well as having an anisotrichosis ratio of 0.2 or greater (20% terminal hair diameter variation or greater) based on trichoscopic measurement wherein one standard deviation of the mean diameter of terminal hairs divided by the mean of terminal hairs was 0.2 or greater (i.e., 20% or greater) as measured by trichoscopic imaging (TrichoSciencePro® with version 1.6 software; Trilogic LLC, Boston, MA) at 25× magnification within a measurement area of 83 sq. mm at either mid-parting area 3 cm from anterior hairline of frontal scalp or vertex mid-part section of scalp based on the area of maximum visible hair thinning. The seven female subjects that met the criteria for FPHL were required to use the composition described in Table 1B (formula JPM-01-158) for 6 months of daily application of 1 mL of product applied to the scalp.

Test subjects were instructed to spray the treatment composition according to an embodiment of the disclosure as set forth in Table 1B to all areas of the scalp by spraying around 0.25 mL of the composition to each quadrant of the scalp: right front & mid scalp, right rear scalp and crown, left front and mid scalp, and left rear scalp and crown, at a total dose of around 1 mL (around 0.9 gram) per approximately 700 cm² scalp surface area once daily over a treatment period of around 24 weeks (or around 6 months or 180±20 days) with clinical and trichoscopic measurements made at the end of a first part of the treatment period (at approximately 12 weeks or 3 months) and at the end of the full treatment period (at approximately 24 weeks or 6 months). Each subject reported having applied a dose of the treatment composition once daily as instructed over the course of the full treatment period. The actual treatment period varied slightly from subject to subject, ranging from 82-107 days for the first part of the treatment period (around three months) and from 168-205 days over the entire treatment period (around 6 months). Treatment applications for the seven test subjects averaged 88 applications in the first part of the treatment period and 195 applications over the full treatment period. Treatment ended at around 24 weeks (6 months), with post-treatment clinical exams and trichoscopic measurements made at approximately 30 weeks (6 weeks after the last treatment, 30 weeks from start) and at approximately 42 weeks (18 weeks after the last treatment, 42 weeks from start) to determine if there was any regression of the improvements seen. There was no regression—the mean density and diameters of terminal hairs did not regress and vellus hair counts did not increase. The following results were observed:

Among all seven subjects, average terminal hair density increased after 90 days ±10 days of treatment by 18.7% (p<0.05; small sample t-test). Among all seven subjects, average terminal hair density increased after 180 days ±20 days of treatment by 29% (p<0.05; small sample t-test). Among all seven subjects, average terminal hair diameter increased after 90 days ±10 days of treatment by 6.3% (p<0.05; small sample t-test). Among all seven subjects, average terminal hair diameter increased after 180 days ±20 days of treatment by 16.2% (p<0.01; small sample t-test).

Among all seven subjects, average vellus hair density decreased after 90 days ±10 days of treatment by 55.4% (p<0.01; small sample t-test).

Among all seven subjects, average vellus hair density decreased after 180 days ±25 days of treatment by 73.8% (p<0.01; small sample t-test).

Among all seven subjects, average ratio of terminal hairs to vellus hairs were: 4.8:1 at baseline; 12.7:1 at 90 days of treatment, and 21.5:1 at 180 days of treatment.

Among all seven subjects, the analysis of average terminal hair density and average terminal hair diameter at 222±20 days of treatment and 6 weeks after the end of treatment and at 300±20 days (or around 18 weeks after the end of treatment) show that the improvements in hair density and diameter did not regress back to the baseline values of average hair diameter and density. In fact, improvements in average diameter and density at the end of treatment (180±20 days) were maintained or somewhat improved at 18 weeks after treatment had stopped. Regression back to the baseline condition is an established problem with prior art treatments for hair loss, such as minoxidil and finasteride. Individual results are shown below in Tables 5-7.

TABLE 5
Terminal Hair Density (terminal hairs/cm²)*

| | | | | 222 days ± | 300 days ± |
|---|---|---|---|---|---|
| | | | | 25 days | 25 days |
| | | | | (around | (around |
| | | | | 6 weeks | 18 weeks |
| | Base- | 90 days ± | 180 days ± | after end of | after end of |
| Subject | line | 10 days | 20 days | treatment) | treatment) |
| 001 | 28 | 44 | 56 | 48 | 60 |
| 002 | 38 | 54 | 54 | 72 | 83 |
| 003 | 65 | 66 | 70 | 73 | 60 |
| 004 | 38 | 42 | 47 | 38 | 48 |
| 005 | 46 | 55 | 50 | 48 | 53 |
| 006 | 44 | 50 | 50 | 47 | 53 |
| 007 | 52 | 58 | 67 | 54 | 71 |

*Density is hair count per area. The trichoscope measures 83 mm² which is converted to number of terminal hairs (hairs >40 microns) per cm².

TABLE 6
Average Terminal Hair Diameter (in microns)*

| | | | | 222 days ± | 300 days ± |
|---|---|---|---|---|---|
| | | | | 25 days | 25 days |
| | | | | (around | (around |
| | | | | 6 weeks | 18 weeks |
| | Base- | 90 days ± | 180 days ± | after end of | after end of |
| Subject | line | 10 days | 20 days | treatment) | treatment |
| 001 | 76 | 82 | 89 | 79 | 96 |
| 002 | 79 | 76 | 77 | 79 | 93 |
| 003 | 100 | 103 | 121 | 110 | 124 |
| 004 | 69 | 70 | 83 | 79 | 93 |
| 005 | 78 | 88 | 98 | 105 | 109 |
| 006 | 76 | 82 | 80 | 93 | 119 |
| 007 | 77 | 89 | 97 | 112 | 97 |

*Mean diameter of terminal hairs (being 40 microns or larger) based on actual hairs measured in 83 mm² area using trichoscope.

TABLE 7
Vellus Hair Density (hairs <40 u/cm²)*

| | | | | 222 days ± | 300 days ± |
|---|---|---|---|---|---|
| | | | | 25 days | 25 days |
| | | | | (around | (around |
| | | | | 6 weeks | 18 weeks |
| | Base- | 90 days ± | 180 days ± | after end of | after end of |
| Subject | line | 10 days | 20 days | treatment) | treatment) |
| 001 | 6 | 0 | 11** | 2 | 4 |
| 002 | 11 | 7 | 0 | 0 | 0 |
| 003 | 5 | 0 | 0 | 0 | 0 |
| 004 | 7 | 4 | 0 | 0 | 0 |
| 005 | 12 | 5 | 0 | 1 | 0 |
| 006 | 11 | 6 | 4 | 0 | 2 |
| 007 | 13 | 7 | 2 | 0 | 0 |

*Density is hair count per area. The trichoscope measures 83 mm² which is converted to number of vellus hairs (hairs <40 microns) per cm².
**Outlier; potential measurement error

As can be seen, application of a treatment composition according to an embodiment herein and according to a method herein results in significant improvements in average terminal hair density and average terminal hair diameter after 90 days treatment and even greater improvements at 180 days. As the hair area in this Example was a scalp, where it is desired to have terminal hair and not vellus hair, the reduction in vellus hair over the two treatment periods is also noted. Vellus hairs are very thin and friable non-pigmented hairs with a diameter less than 40 microns. As the percentage of vellus hairs decrease, with no decrease in the average diameter of terminal hairs, the total average hair thickness increases.

In addition to the dramatic reduction in the mean vellus hair density after 90 and 180 days of treatment of the Table 1B composition, a trichoscopic analysis of changes in “thin” terminal hairs (40-80u diameters), “normal” terminal hairs (80-110u diameters) and “thick” terminal hairs (>110u diameters) was made by comparing trichoscopic data for the 7 subjects between baseline and after 90 and 180 days of treatment. The analysis revealed that the change in “hair thickness”, with hair thickness defined as the average density of normal plus thick hairs, increased 35% between baseline and 90 days and increased 45% between baseline and 180 days.

### Example 2—Biopsy Study for Immune Response

Three otherwise healthy female adults with FPHL, who were initially assessed as being Sinclair (grade II-V) or Ludwig (grade I-Ill) classes of hair loss by a dermatologist, were enrolled in a six-month study. Each of the three participants was confirmed as having 20% or more anisotrichosis (ratio of one standard deviation of terminal hair diameters to the mean of terminal hair diameters) based on trichoscopy. Trichoscopic evaluation was conducted using images with 25× optical magnification from Firefly® Digital Trichoscope DE330T and analyzed with Trichoscience Pro software at either (a) mid-part 3 cm from anterior hairline of frontal scalp or b) vertex mid-part section based on area of maximum hair thinning.

Each participant applied approximately 0.0013 g/cm² of scalp treatment area with a treatment composition according to an embodiment of the disclosure as set forth in Table 1B once daily for a treatment period of six months. Treatment compliance was assessed every six weeks by weight check of previously weighed spray dispensing unit containing a predetermined amount of the Table 1B treatment composition. The treatment composition used was a leave-in treatment applied after shampooing (if the subject shampooed on a given day). Punch biopsy tissue specimens (3 mm) were taken from the area of scalp exhibiting FPHL of each subject/participant one day prior to commencing the treatment period (pre-treatment/baseline) and at the end of the six-month treatment period (end of treatment). Each biopsy specimen was flash-frozen and stored at minus 20° C. RNA was extracted from frozen biopsy tissues and RNA-Seq data analysis (Azenta Genewiz; now Azenta Life Sciences; USA, Inc. Burlington, MA) was performed. Differential gene expression was determined from both pre-treatment/baseline and end of treatment tissue samples using next-generation sequencing by Azenta NGS Division of Azenta Life Sciences (south Plainfield, N.J.). A significant change in gene expression was defined as Log 2 fold change of greater than 1, with a p value of <0.05 or less. Table 8 below shows the differentially regulated immunoglobulins and chemokines per subject at the end of the treatment period.

TABLE 8
Differentially Regulated Immunoglobulins
& affected immunomodulator genes

| | Subject 1 | Subject 2 | Subject 3 |
|---|---|---|---|
| IGHV3-7 | −2.3 | | −2.3 |
| IGHV3-30 | | −3.9 | |
| IGHV3-48 | −2.2 | | −2.2 |
| IGHV3-53 | | | −6.1 |
| IGKV2D-28 | | | −3.0 |
| IGKV3-11 | | −3.8 | |
| IGKV3-20 | | −2.9 | |
| IGKVID-16 | −5.4 | | |
| IGLV1-40 | | −3.6 | −3.6 |
| IGLV1-51 | | | −2.5 |
| IGLV2-8 | −2.9 | | |
| IGLV2-11 | | | −3.0 |
| IGLV2-23 | | | +2.3 |
| IGLV3-1 | | −3.2 | |
| IGLV3-19 | +2.0 | | −5.4 |
| CCL24 | | −2.0 | |
| CXCL10 | | | −2.1 |
| CXCL13 | −2.9 | | |
| PRSS21 | | −2.0 | |

The differential analysis of the biopsy tissue at the end of the treatment period compared to just prior to treatment indicates an epigenetic end effect of how the body responds to the treatment composition by measuring how the genomic profile of the target tissue has changed based on the treatment. After the 6 month treatment period in this example, the differential analysis indicates that a range of genes (mostly immunoglobulins) are significantly lower than at the start of treatment. This data shows a decrease in a range of immunoregulatory antibody proteins, which, accompanied by the phenotypic changes seen (increase in hair density and diameter), indicate that certain antigenic changes associated with HHT, FPHL and MPHL may cause upregulation of immunoglobulins and chemokines that are subsequently lowered by application of the treatment composition over the treatment period. It also may indicate that FPHL among these test subject described, is likely caused by immunoregulated event(s) and may be related to a change in the hair follicle immune privilege normally in place in a portion of the anagen hair cycle.

According to still other embodiments, treatment compositions comprise ingredients and amounts according to one or more of the following Embodiments ands methods of treating age-related human hair thinning, FPHL, and/or MPHL comprise steps according to one or more of the following Embodiments:

Embodiment 1. A composition for treating age-related human hair thinning, the composition comprising one or more active ingredients useful in increasing the density or the diameter of terminal hairs and a carrier; wherein the one or more active ingredients comprises 0.001% to 0.1% of a copper chlorin by weight of the composition.

Embodiment 2. The composition of embodiment 1 wherein the copper chlorin is one or more of a sodium copper chlorophyllin complex, trisodium copper chlorin e6, or disodium copper isochlorin e4.

Embodiment 3. The composition of any one of embodiments 1-2 wherein the composition downregulates the expression at least one gene, the at least one gene comprising IGKV1 D-16; IGKV3-20; IGKV3-11; IGKV2D-28; IGKV2D-28; IGLV3-19; IGLV1-40; IGLV1-51; IGLV2-11; IGLV1-51; IGHV3-48; IGHV3-30; IGHV3-53; IGHV3-7; PRSS21; GZMK; ICOS; CXCL13; CXCL10; or CCL24.

Embodiment 4. The composition of embodiment 3 wherein the composition downregulates the expression of the at least one gene by at least 200%.

Embodiment 5. The composition of any one of embodiments 1-4 wherein the composition upregulates the expression of IGLV2-8, or IGLV2-23, or both.

Embodiment 6. The composition of embodiment 5 wherein the composition upregulates the expression of IGLV2-8 or IGLV2-23 by at least 200%.

Embodiment 7. The composition of any one of embodiments 1-6 wherein the copper chlorin comprises (1) at least 30% by weight disodium copper isochlorin e4 or trisodium copper (II) chlorin e6 or a combination thereof and (2) one or more of chlorin e4, isochlorin e4, copper chlorin e4, copper isochlorin e4, chlorin e6, copper chlorin p6, copper pheophorbide a, copper pyropheophorbide a, copper purpurin 7, copper rhodin g7, copper rhodochlorin or oxidized forms or salts thereof.

Embodiment 8. The composition of any one of embodiments 1-7 wherein the carrier comprises alcohol and water.

Embodiment 9. A method of treating human hair thinning, the method comprising topically applying a dose of a treatment composition comprising one or more active ingredients useful in increasing the density or the diameter of terminal hairs and a carrier; wherein the one or more active ingredients comprises 0.001% to 0.1% of a copper chlorin by weight of the treatment composition to a area of human hair experiencing hair thinning resulting from aging.

Embodiment 10. The method of embodiment 9 wherein the treatment composition is in a liquid form and the dose is around 0.001 grams to around 0.002 grams per cm2 of the area of human hair.

Embodiment 11. The method of embodiment 9 wherein the dose is around 0.3 grams to around 2 grams per 700 cm2 of the area or human hair.

Embodiment 12. The method of any one of embodiments 9-11 wherein the dose comprises around 20 to around 1000 μg of the copper chlorin.

Embodiment 13. The method of any one of embodiments 9-12 wherein the copper chlorin comprises one or more of copper chlorin e6, copper isochlorin e4, copper chlorin p6, copper rhodin g7, or copper chlorophyllin complex.

Embodiment 14. The method of any one of embodiments 9-13 wherein the copper chlorin comprises a sodium salt of one or more of copper chlorin e6, copper isochlorin e4, copper chlorin p6, copper rhodin g7, or copper chlorophyllin complex.

Embodiment 15. The method of any one of embodiments 9-14 wherein the copper chlorin comprises (1) at least 30% by weight disodium copper isochlorin e4 or trisodium copper (II) chlorin e6 or a combination thereof and (2) one or more of chlorin e4, isochlorin e4, copper chlorin e4, copper isochlorin e4, chlorin e6, copper chlorin p6, copper pheophorbide a, copper pyropheophorbide a, copper purpurin 7, copper rhodin g7, copper rhodochlorin or oxidized forms or salts thereof.

Embodiment 16. The method of any one of embodiments 9-15, wherein within 24 hours after topically applying the dose, an expression of one or more genes is upregulated or downregulated by 200% or more, the one or more genes comprising one or more of ARNTL2; BAMBI; CCL20; CSF1; CSF2; CSF3; CTSB; CXCL1; CXCL11; CXCL2; CXCL3; CXCL8; DDX58; DDX60; DDX60L; DKK1; EGR1; EIF2AK2; EPST11; EREG; FGF2; FN1; FOS; GAB1; GADD45B; GDF15; GPX2; HAS3; HBEGF; HERC5; HERC6; HIF1AN; HSP90B1; CMPK2; ICAM1; IFI27; IFI44; IFI44L; IFI6; IFIH1; IFIT1; IFIT2; IFIT5; IGFBP3; IL1A; IGHA1; IL1B; IL6; JUN; JUNB; LY6E; MAFF; MMP8; MYC; NAB1; FGF2; OAS2; OAS3; OASL; OVOL1; PARP 9; PARP12; PARP14; PCDH1; PLAUR; PLSCR1; RSAD2; PTGFRN; PTGS2; RHOB; RHOB; RTP4; SAMD9L; SERPINB2; TGFB1; SERPING1; SIGLEC1; SKP2; SPATS2L; STAT1; TGFB2; TGFB2-OT1; TNF; TNFAIP6; TNFSF12; SOD2; USP18; USP41; VEGFA; and XAF1.

Embodiment 17. The method of any one of embodiments 9-15, wherein within 24 hours after topically applying the dose, an expression of one or more genes is upregulated or downregulated by 200% or more, the one or more genes comprising one or more of ARRDC3, BAMBI, CXCL10, DEFB1; EPGN; FOS; HBEGF; IFI44L; MAFF; PLAUR; PPP1R15A; PTGS2; SPRR2B; TNFAIP3; UBE20; and VEGFA.

Embodiment 18. The method of any one of embodiments 9-17 further comprising repeating the applying step at least once daily for a period of consecutive days in a treatment period.

Embodiment 19. The method of embodiment 18 wherein the treatment period is at least 80 days.

Embodiment 20. The method of any one of embodiments 18-19 wherein the period of consecutive days is at least 80 days.

Embodiment 21. The method of any one of embodiments 18-20 wherein the treatment period is at least 160 days.

Embodiment 22. The method of any one of embodiments 18-21 wherein, when measured at an end of the treatment period, an expression of one or more genes is downregulated, the one or more genes comprising one or more of IGKV1 D-16; IGKV3-20; IGKV3-11; IGKV2D-28; IGKV2D-28; IGLV3-19; IGLV1-40; IGLV1-51; IGLV2-11; IGLV1-51; IGHV3-48; IGHV3-30; IGHV3-53; IGHV3-7; PRSS21; GZMK; ICOS; CXCL13; CXCL10; and CCL24.

Embodiment 23. The method of embodiment 22 wherein the expression is downregulated by at least 200%.

Embodiment 24. The method of any one of embodiments 9-23 wherein, when measured at an end of the treatment period, an expression of IGLV2-8 or IGLV2-23 or both is upregulated.

Embodiment 25. The method of any one of embodiments 9-24 wherein, when measured at an end of the treatment period, an expression of at least three or more genes are downregulated, the three of more genes comprising three or more of IGKV1D-16; IGKV3-20; IGKV3-11; IGKV2D-28; IGKV2D-28; IGLV3-19; IGLV1-40; IGLV1-51; IGLV2-11; IGLV1-51; IGHV3-48; IGHV3-30; IGHV3-53; IGHV3-7; PRSS21; GZMK; ICOS; CXCL13; CXCL10; and CCL24.

Embodiment 26. The method of any one of embodiments 9-25 wherein the treatment composition is according to any one of embodiments 1-8.

As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated.

As used herein, the terms “treat,” “treating,” “treatment,” and the like may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition. The term “treat” and synonyms contemplate administering a therapeutically effective amount of a composition of the disclosure to an individual in need of such treatment. Within the meaning of the disclosure, “treatment” also includes relapse prophylaxis or phase prophylaxis, as well as the treatment of acute or chronic signs, symptoms and/or malfunctions. The treatment can be orientated symptomatically, for example, to suppress symptoms. It can be affected over a short period, be oriented over a medium term, or can be a long-term treatment, for example within the context of a maintenance therapy.

As used herein, the terms “prevent,” “preventing,” and “prevention,” are art-recognized, and when used in relation to a condition, such as HHT, AGA or FPHL and MPHL, is well understood in the art, and includes administration of a composition which reduces the frequency of, or delays the onset of, symptoms of a medical condition in a subject relative to a subject which does not receive the composition. Thus, prevention of such as HHT, AGA, or FPHL and MPHL includes the topical application of a treatment composition herein on areas of skin or scalp that may already show signs of HHT, AGA or FPHL and MPHL or that may otherwise develop signs of HHT, AGA, or FPHL and MPHL if left untreated.

In some cases, the compositions and methods disclosed herein comprise those for treating or preventing HHT, AGA, or FPHL and MPHL. In some cases, the compositions and methods disclosed herein comprise those for treating HHT, AGA or FPHL and MPHL. In some cases, the compositions and methods disclosed herein comprise those for preventing HHT, AGA or FPHL and MPHL.

All numerical values, ratios, or percentages indicated herein as a range include each individual amount, numerical value, or ratio within those ranges and any and all subset combinations within ranges, including subsets that overlap from one preferred range to a more preferred range. References to “about” or “around” with respect to numerical values generally mean (1)+/−1 for values expressed as whole numbers (without a decimal, e.g., around 15% means 14-16%); (2)+/−0.1 for values expressed with a single decimal place (for example, around 9.5% means 9.4-9.6%; and (3)+/−0.01 for values expressed with two or more decimal places (for example, around 0.02 means 0.01-0.03, each of the foregoing excluding values that would result in a negative number.

References herein to “at least one” means one or more and includes individual components as well as mixtures/combinations.

All numerical values, ratios, or percentages used in describing ingredients are to be understood as being modified in all instances by the term “about.” References to “about” or “around” with respect to numerical values generally mean (1)+/−1 for values expressed as whole numbers (without a decimal, e.g., around 15% means 14-16%); (2) +/−0.1 for values expressed with a single decimal place (for example, around 9.5% means 9.4-9.6%; and (3)+/−0.01 for values expressed with two or more decimal places (for example, around 0.02 means 0.01-0.03, each of the foregoing excluding values that would result in a negative number.

All numerical values, ratios, or percentages indicated herein as a range include each individual amount, numerical value, or ratio within those ranges and any and all subset combinations within ranges, including subsets that overlap from one preferred range to a more preferred range. For example, a range from 1-5, includes the individual values of 1, 1.4, 2, 2.8, 3, 4, 4.5, and 5, as well as subranges such as 2-5, 1.6-3.3, 3-5, 2-3, 2-4, 1-4, etc. Unless otherwise indicated, percentages, parts and ratios are to be understood as based upon the total weight of the chelated copper chlorin compound or a treatment composition herein.

Any treatment composition ingredient, other than a chlorin, described herein as included or optional in any embodiment herein may also be excluded from any embodiment herein. Unless explicitly excluded herein, any ingredients in any treatment composition embodiment and/or method steps described herein may be used with any other embodiment, even if not specifically described herein with that particular embodiment. Any treatment composition embodiment herein may comprise, consist essentially of, or consist of any combination of ingredients described herein.

References herein to water (without any modifier) include potable water, distilled water, deionized water, or other forms of purified, filtered, or cleaned water suitable for use in topical skin treatment compositions and intradermal treatments. These forms of water may be substituted for references herein to deionized water, other than in the claims.

The preceding examples illustrate various aspects and preferred embodiments of the invention but are not intended to be limiting in any way. While particular embodiments of the present invention have been illustrated and described, those skilled in the art will understand that changes and modifications can be made without departing from the spirit and scope of the invention. Accordingly, all changes and modifications that are within the scope of this invention are intended to be covered in the appended claims.

We claim:

1. A composition for treating age-related human hair thinning, the composition comprising one or more active ingredients useful in increasing the density or the diameter of terminal hairs and a carrier;
wherein the one or more active ingredients comprises 0.001% to 0.1% of a copper chlorin by weight of the composition.

2. The composition of claim 1 wherein the copper chlorin is one or more of a sodium copper chlorophyllin complex, trisodium copper chlorin e6, or disodium copper isochlorin e4.

3. The composition of claim 2 wherein the composition downregulates the expression at least one gene, the at least one gene comprising IGKV1D-16; IGKV3-20; IGKV3-11; IGKV2D-28; IGKV2D-28; IGLV3-19; IGLV1-40; IGLV1-51; IGLV2-11; IGLV1-51; IGHV3-48; IGHV3-30; IGHV3-53; IGHV3-7; PRSS21; GZMK; ICOS; CXCL13; CXCL10; or CCL24.

4. The composition of claim 3 wherein the composition downregulates the expression of the at least one gene by at least 200%.

5. The composition of claim 2 wherein the composition upregulates the expression of IGLV2-8, or IGLV2-23, or both.

6. The composition of claim 5 wherein the composition upregulates the expression of IGLV2-8 or IGLV2-23 by at least 200%.

7. The composition of claim 1 wherein the copper chlorin comprises (1) at least 30% by weight disodium copper isochlorin e4 or trisodium copper (II) chlorin e6 or a combination thereof and (2) one or more of chlorin e4, isochlorin e4, copper chlorin e4, copper isochlorin e4, chlorin e6, copper chlorin p6, copper pheophorbide a, copper pyropheophorbide a, copper purpurin 7, copper rhodin g7, copper rhodochlorin or oxidized forms or salts thereof.

8. The composition of claim 1 wherein the carrier comprises alcohol and water.

9. A method of treating human hair thinning, the method comprising topically applying a dose of a treatment composition comprising one or more active ingredients useful in increasing the density or the diameter of terminal hairs and a carrier;
wherein the one or more active ingredients comprises 0.001% to 0.1% of a copper chlorin by weight of the treatment composition to a area of human hair experiencing hair thinning resulting from aging.

10. The method of claim 9 wherein the treatment composition is in a liquid form and the dose is around 0.001 grams to around 0.002 grams per cm² of the area of human hair.

11. The method of claim 9 wherein the dose is around 0.3 grams to around 2 grams per 700 cm² of the area or human hair.

12. The method of claim 9 wherein the dose comprises around 20 to around 1000 μg of the copper chlorin.

13. The method of claim 12 wherein the copper chlorin comprises one or more of copper chlorin e6, copper isochlorin e4, copper chlorin p6, copper rhodin g7, or copper chlorophyllin complex.

14. The method of claim 9 wherein the copper chlorin comprises a sodium salt of one or more of copper chlorin e6, copper isochlorin e4, copper chlorin p6, copper rhodin g7, or copper chlorophyllin complex.

15. The method of claim 9 wherein the copper chlorin comprises (1) at least 30% by weight disodium copper isochlorin e4 or trisodium copper (II) chlorin e6 or a combination thereof and (2) one or more of chlorin e4, isochlorin e4, copper chlorin e4, copper isochlorin e4, chlorin e6, copper chlorin p6, copper pheophorbide a, copper pyropheophorbide a, copper purpurin 7, copper rhodin g7, copper rhodochlorin or oxidized forms or salts thereof.

16. The method of claim 9, wherein within 24 hours after topically applying the dose, an expression of one or more genes is upregulated or downregulated by 200% or more, the one or more genes comprising one or more of ARNTL2; BAMBI; CCL20; CSF1; CSF2; CSF3; CTSB; CXCL1; CXCL11; CXCL2; CXCL3; CXCL8; DDX58; DDX60; DDX60L; DKK1; EGR1; EIF2AK2; EPST11; EREG; FGF2; FN1; FOS; GAB1; GADD45B; GDF15; GPX2; HAS3; HBEGF; HERC5; HERC6; HIF1AN; HSP90B1; CMPK2; ICAM1; IFI27; IFI44; IFI44L; IFI6; IFIH1; IFIT1; IFIT2; IFIT5; IGFBP3; IL1A; IGHA1; IL1B; IL6; JUN; JUNB; LY6E; MAFF; MMP8; MYC; NAB1; FGF2; OAS2; OAS3; OASL; OVOL1; PARP 9; PARP12; PARP14; PCDH1; PLAUR; PLSCR1; RSAD2; PTGFRN; PTGS2; RHOB; RHOB; RTP4; SAMD9L; SERPINB2; TGFB1; SERPING1; SIGLEC1; SKP2; SPATS2L; STAT1; TGFB2; TGFB2-OT1; TNF; TNFAIP6; TNFSF12; SOD2; USP18; USP41; VEGFA; and XAF1.

17. The method of claim 9 wherein within 24 hours after topically applying the dose, an expression of one or more genes is upregulated or downregulated by 200% or more, the one or more genes comprising one or more of ARRDC3, BAMBI, CXCL10, DEFB1; EPGN; FOS; HBEGF; IFI44L; MAFF; PLAUR; PPP1R15A; PTGS2; SPRR2B; TNFAIP3; UBE20; and VEGFA.

18. The method of claim 15 further comprising repeating the applying step at least once daily for a period of consecutive days in a treatment period.

19. The method of claim 18 wherein the treatment period is at least 80 days.

20. The method of claim 18 wherein the period of consecutive days is at least 80 days.

21. The method of claim 18 wherein the treatment period is at least 160 days.

22. The method of claim 18 wherein, when measured at an end of the treatment period, an expression of one or more genes is downregulated, the one or more genes comprising one or more of IGKV1D-16; IGKV3-20; IGKV3-11; IGKV2D-28; IGKV2D-28; IGLV3-19; IGLV1-40; IGLV1-51; IGLV2-11; IGLV1-51; IGHV3-48; IGHV3-30; IGHV3-53; IGHV3-7; PRSS21; GZMK; ICOS; CXCL13; CXCL10; and CCL24.

23. The method of claim 22 wherein the expression is downregulated by at least 200%.

24. The method of claim 22 wherein, when measured at an end of the treatment period, an expression of IGLV2-8 or IGLV2-23 or both is upregulated.

25. The method of claim 18 wherein, when measured at an end of the treatment period, an expression of at least three or more genes are downregulated, the three of more genes comprising three or more of IGKV1 D-16; IGKV3-20; IGKV3-11; IGKV2D-28; IGKV2D-28; IGLV3-19; IGLV1-40; IGLV1-51; IGLV2-11; IGLV1-51; IGHV3-48; IGHV3-30; IGHV3-53; IGHV3-7; PRSS21; GZMK; ICOS; CXCL13; CXCL10; and CCL24.
